(19) United States
(12) Patent Application Publication
(10) Pub. No.: US 2013/0167840 A1
(43) Pub. Date: Jul. 4, 2013

(54) CO2 SORBENT COMPOSITION WITH O2 CO-GENERATION

(71) Applicants: Bruce F. Monzyk, Town Creek, AL (US); Chad M. Cucksey, Worthington, OH (US); Timothy S. Rennick, Columbus, OH (US); Brian J. Sikorski, Plain City, OH (US); Martha W. McCauley, Powell, OH (US)

(72) Inventors: Bruce F. Monzyk, Town Creek, AL (US); Chad M. Cucksey, Worthington, OH (US); Timothy S. Rennick, Columbus, OH (US); Brian J. Sikorski, Plain City, OH (US); Martha W. McCauley, Powell, OH (US)

(73) Assignee: BATTELLE MEMORIAL INSTITUTE, Columbus, OH (US)

(21) Appl. No.: 13/812,901

(22) PCT Filed: Aug. 2, 2011
(86) PCT No.: PCT/US11/46343

§ 371 Date: Mar. 21, 2013

Related U.S. Application Data
(60) Provisional application No. 61/369,805, filed on Aug. 2, 2010.

(51) Int. Cl. B01J 20/06; B32B 9/00; A62B 23/02; A62B 21/00; A61M 16/22; B32B 3/26; A62B 7/08

(52) U.S. Cl. CPC B01J 20/06; B32B 3/266; B32B 9/00; B32B 3/26; A62B 7/08; A62B 21/00; A61M 16/22; A62B 23/02
USPC 128/202.26; 423/594.1; 252/184; 428/402; 428/64.1; 442/121; 442/1; 428/311.11; 428/319.1; 428/137; 428/697; 428/188

(57) ABSTRACT

The invention provides for a sorbent composition comprising Fe(IV), Fe(V), Fe(VI), and/or a mixture of thereof. (“the ferrate compound”), wherein upon exposure to CO₂ and moisture, the sorbent composition absorbs CO₂ and co-generates O₂, and materials, systems and methods of using this sorbent composition.

### FIELD OF THE INVENTION

The invention provides for the application of ferrate(IV), ferrate(VI) ferrate(V), or a mixture thereof as a CO₂ sorbent composition while co-generating O₂.

### BACKGROUND OF THE INVENTION

CO₂ sorbents are needed in many situations, such as scuba diving, ambulances, fire fighting, mining, sleep deprivation devices, and other emergency situations such as caved-in mines, poison gas leaks etc. In these situations, the amount of portable O₂ is generally very limited. Moreover, current CO₂ sorbents, which are mostly based on soda lime, are capable of picking up 25 vol/vol % to 35 vol/vol % in CO₂ at most. Therefore, it is desirable to have CO₂ sorbents that can co-generate O₂ and have a high CO₂ absorbing capacity. In some situations, such as underwater rebreather and space suits, it is also desirable to have a CO₂ sorbent that can reduce the relative humidity of the gas, which will enable the diver or astronaut to breathe more comfortably.

Ferrate(VI) is known to generate O₂. In addition, Tsapin et al. teach that ferrate(VI) produces CO₂ in some situations in his published article titled “Ferrate(VI) as a Possible Oxidant on the Martian Surface” (collaborated for NASA: http://trs-w.jpl.nasa.gov/dspace/bitstream/2014/18372/1/99-849.pdf).

US patent application publication 2009/0061267 and PCT published application WO/2007/027876 to Monzyk et al. disclose a “Power Device and Oxygen Generator”. In particular, a system for oxygen, hydrogen and carbon mass regeneration and recycling for breathing are disclosed, in which CO₂ and/or H₂O are photolytically converted to a form of energy using a catalyst and O₂. Further, blends of ferrate(VI) can be used as a battery, along with CO₂, in this energy conversion. Monzyk et al. do not disclose or suggest that ferrate(VI) absorbs CO₂. More importantly, according to Monzyk et al., photolytically provided energy, in the forms of electrons and hydrogen ions, are needed for this energy conversion and CO₂ recycling. However, in emergency and other situations where it is impossible or difficult to obtain photolytical energy, such as underwater situations and/or the dark environment of traveling vessels and mines, it is desirable to use a CO₂ sorbent material to absorb CO₂ without having to provide energy photolytically.

There have been a few rebreather designs, which have a CO₂ sorbent material that produces oxygen as it absorbs carbon dioxide, such as the Oxylite with potassium superoxide: 4KO₂+2CO₂→2K₂CO₃+3O₂. However, this rebreather system is dangerous because of the explosively hot reaction that happens if water gets on the potassium superoxide. Therefore, it is desirable to have a stable rebreather system with an absorbent material that is capable of absorbing CO₂ and co-generating O₂. Such a system would not expose a user to any risk of water produced explosive hot reactions.

### BRIEF DESCRIPTION OF THE INVENTION

There exists a need for a sorbent composition that can absorb CO₂ and co-generate O₂ in a hostile environment. A first broad embodiment of the invention provides for a sorbent composition comprising Fe(VI), Fe(V), Fe(VI), and/or a mixture thereof (hereafter called “ferrate” or “ferrate compound”), wherein upon exposure to CO₂ and H₂O, the sorbent composition is capable of absorbing CO₂ and co-generating O₂.

According to some embodiments of the present invention, the sorbent composition is in the form of granule, extrudate, sphere, disk, briquette, pellet, prill, solid solution, microsphere, encapsulate, or a mixture thereof. Preferably, the sorbent composition includes one or more hygroscopic materials. More preferably, it includes H₂O.

Further, the sorbent composition comprises one or more cooling agents. The cooling agents can be used to control temperature so as to enable the formation of a liquid water layer on the sorbent composition, which is needed for the CO₂ absorption and O₂ generation.

Some embodiments of the present invention provide for a sorbent material suitable for removal of CO₂ and co-generation of O₂, which comprises one or more sorbent compositions described above. Preferably, the sorbent compositions are embedded in one or more fibers.

In some further embodiments, the sorbent material includes a sorbent layer formed by one or more sorbent compositions joining with one or more substrates. Preferably, the sorbent compositions are coated on one or more substrates. Suitable substrates include one or more mats, beads, screens, porous material (paper, fabric or plastic), perforated plastic, perforated and corrugated plastic, woven fabric, non-woven fabric, or mixtures thereof.

Preferably, the substrate comprises one or more hygroscopic materials. The hygroscopic materials can be deliquescent, or otherwise absorb and release water as needed. For example, the hygroscopic material can absorb and store water, and then when the humidity is reduced, the stored water can be released. Unlimited examples of the hygroscopic material include clay, molecular sieve, and gel.

In some further embodiments of the sorbent material, the substrate comprises a top layer and a bottom layer; one or more sorbent compositions form a sorbent layer; and the top layer covers one surface of the sorbent bed and the bottom layer covers the other surface of the sorbent bed. Preferably the top layer has an upper covering, one or more air spacers and a lower covering in contact with an upper surface of the sorbent bed. The air spacers separate the upper covering from the lower covering, forming channels inside the top layer. Preferably, the bottom layer has an upper covering in contact with a lower surface of the sorbent bed, a lower covering, and one or more air spacers separating the upper covering from the lower covering, forming channels inside the top layer.

More preferably, the upper coverings, the air spacers, and the lower coverings of the top layer and the bottom layer comprise one or more porous materials. The suitable porous material comprises matt, screen, porous paper, woven/nonwoven fabric, perforated plastic, or a mixture thereof.

Preferably, the sorbent compositions in the sorbent materials disinfect the incoming air and/or the revitalized air.

Alternatively, some embodiments of the present invention provide for a breathing system for use in a hostile environment to absorb CO₂ and co-generate O₂, comprising

(a) one or more breathing components to receive one or more exhaled moist air streams from one or more users, wherein the exhaled moist air stream comprises CO₂ and moisture; and

(b) one or more sorption components for absorbing CO₂ and H₂O, and to co-generate O₂, resulting in solid products and a revitalized air suitable for rebreathing, wherein the sorption component comprises one or more sorbent materials described above.

Preferably, the breathing system includes one or more cooling agents and/or cooling components. In some embodiments, the breathing system includes at least one component comprising H₂O.

Preferably, the breathing system includes one or more agitation components to shake loose the solid products from the sorbent material.

The breathing system can also include at least one pump to drive the incoming air stream through the sorbent layer, wherein the pump comprises an air pump, a vacuum pump, or a similar device. At least one suitable exit gas filtration component can also be included so as to prevent any fines or dust from exiting with the revitalized air.

The above breathing system can be used as a rebreather underwater, as emergency first responders, in mining, and in other emergency situations.

Preferably, the breathing system is portable.

Alternatively, some embodiments of the present invention provide for a method for absorbing CO₂ and co-generating O₂, comprising the steps of:

(a) providing one or more breathing systems of claims 17 to 24;

(b) introducing one or more streams of moist air containing CO₂ and H₂O into the breathing system; and

(c) contacting the sorbent material with H₂O to condense on and/or be absorbed into the sorbent material, wherein a part or all of the ferrate compounds dissolve in H₂O to absorb CO₂ and to co-generate O₂, resulting in solid products and a revitalized air suitable for rebreathing.

Preferably, pH is in a range of about 6 to about 10, more preferably in a range of about 6.5 to about 9, and most preferably in a range of about 7 to about 8.

According to some embodiments of the method, the temperature is controlled to enable or assist in the formation of a liquid water layer on the sorbent material. The temperature control can be achieved through the dual factors of spreading out the ferrate compounds and the addition of the cooling agents.

Preferably, the method includes a step of providing an additional H₂O.

The method can also include a step of shaking loose some or all of the solid products from the sorbent material.

In some embodiments, the method of the present invention includes steps of discharging the revitalized air and/or recirculating the discharged revitalized air.

### BRIEF DESCRIPTION OF THE DRAWINGS

FIG. 1 is a diagram showing a process of an incoming moist air stream containing CO₂ and H₂O flowing through and interacting the ferrate granules in the sorbent material. The incoming air stream first forms a liquid film on the surface of the ferrate granules. Then the ferrate compounds in contact with the liquid film dissolves in the liquid film to form metal and ferrate ions. The resulting free ferrate ions interact with CO₂ and H₂O from the incoming air stream to revitalize the incoming air stream by absorbing CO₂ and co-generating O₂.

FIG. 2A is a perspective view of an embodiment of the sorbent material of the present invention, in which the sorbent material and a substrate can be incorporated into a sheet.

FIG. 2B is a perspective view of an embodiment of the sorbent material of the present invention, in which the sorbent material and a substrate can be incorporated into a spiral.

FIG. 3A is an expanded perspective view of a further embodiment of the sorbent material of the present invention. This embodiment of the sorbent material can be in the form of sheets and/or spiral. Specifically, the sorbent material has three layers, including a top layer, a middle layer and a bottom layer, wherein the top layer covers one surface of the middle layer and the bottom layer covers the other surface of the middle layer; wherein the middle layer is a sorbent bed comprising one or more ferrate sorbent compositions of various embodiments. The top and bottom layers are the substrate. Further, the top and bottom layers both have three components: an upper covering, a lower covering, and corrugated air spacers separating the upper covering from the lower covering, forming channels through which an incoming air stream can flow through to get in touch with the sorbent bed in the middle layer, thereby the sorbent composition, the ferrate particles, can absorb CO₂ and H₂O from the incoming air and co-generate O₂, resulting in a revitalized air suitable for re-breathing. The revitalized air then can exit through channels formed by the air spacers. The channels used by the exiting air stream and the channels used by the incoming air stream can be the same or different.

FIG. 3B illustrates an expanded view of a corner of the bottom layer for the embodiment shown in FIG. 3A, showing a porous upper covering, a lower covering, and a corrugated air spacer forming channels to separate the upper covering and the lower covering.

FIG. 4A illustrates a perspective view of a soda lime sorbent particle in the process of absorbing CO₂ and H₂O, in which a liquid film initially forms on the surface of a lime particle (CaO), and then the CaO expands upon absorbing CO₂ and H₂O from the liquid film.

FIG. 4B illustrates a perspective view of a ferrate particle in a sorbent material of the present invention, showing that a liquid film forms on the surface of the ferrate particle. In the liquid film, while not wishing to be bound by theory, it is currently believed that the ferrate compounds dissolve and dissociate into metal and ferrate ions. The ferrate ions then react with water and CO₂ at the liquid-gas interface of the liquid film to form OH⁻ and solid products, FeOOH fine particles and KHCO₃ crystals.

FIG. 5 is a schematic diagram which illustrates the system used for Examples 1 to 5.

### DETAILED DESCRIPTION OF THE INVENTION AND BEST MODE

Broadly, the present invention provides for a sorbent composition comprising Fe(IV), Fe(V), Fe(VI), and/or a mixture thereof (“ferrate compound”), wherein upon exposure to CO₂ and H₂O, the sorbent composition is capable of absorbing CO₂ and co-generating O₂. At the same time, the ferrate compound reduces humidity in the atmosphere near the sorbent composition. Interestingly, the action of the ferrate compound is regulated by the levels of CO₂ and/or H₂O present in the sorbent composition and/or in the nearby atmosphere.

Typically, “moisture” refers to the presence of water vapor (H₂O in gaseous forms) in the air or from an exhaling breath. In the present application, the words “moisture,” “water,” and “H₂O” refer to H₂O in both liquid and gaseous phases, that is, the liquid water and gaseous water vapor. Humidity is typically used as a term for the amount of water vapor in the air. Many devices can be used to measure and regulate humidity. A device used to measure humidity is called a psychrometer or hygrometer. A humidistat is used to regulate the humidity of a building with a dehumidifier. These can be analogous to a thermometer and thermostat for temperature control.

Preferably, the “H₂O” in the present invention can come from (1) the moisture in the exhaling breaths of one or more users; (2) the water vapor from the nearby air; (3) the optional water component in the sorbent composition; (4) the additional water provided by the optional component; and/or (5) a mixture or combination thereof, or other similar mechanisms. It is important to note that only when “H₂O” forms a liquid water layer on the ferrate sorbent composition/material through either absorption and/or condensation, “H₂O” of the present invention is useful for or accessible to the sorbent composition/material to absorb CO₂ and co-generate O₂, the mechanisms of which are explained in detail below. While not wishing to be bound by theory, it is presently believed that condensation of the water is the one preferred way to provide the liquid water layer for the sorbent composition/material, while one or more hygroscopic materials and/or water components, or other similar mechanisms, can also provide H₂O for the liquid water layer.

According to some embodiments of the present invention, the sorbent composition of the present invention can be a part or all of a sorbent material suitable for removal of CO₂ and co-generation of O₂. The “sorbent composition/material” refers to the sorbent composition and/or sorbent material. The sorbent material of the present invention can be used in many emergency environments, such as underwater, emergency first responder, space station, mining, and other emergency situations. Preferably, the ferrate compound is in the form of granule, extrudate, sphere, disks, briquettes, pellet, prill, solid solution, microsphere, or a mixture thereof.

The sorbent material of the present invention can be used to revitalize one or more streams of incoming moist air, a gas, a feed gas, a foul air, a foul breathing air, an exhaled breath, and/or a foul gas stream etc., all of which contain H₂O and excess CO₂, and all of which are referring to interchangeably as an incoming air stream. Further, a breath, a breathed air or a foul air in the present application is defined as an air stream containing H₂O and excess CO₂. The water in the incoming air stream can come from the moisture in the air stream itself, and moisture and/or H₂O from the surrounding environment. Some of the air streams contain very little H₂O, and thus, additional water needs to be provided to enable the sorbent material to absorb CO₂ and co-generate O₂. Preferably, additional water can come from the existing H₂O in the sorbent composition/component, and/or the additional H₂O component or equipment that can be controlled to supply additional H₂O to the sorbent materials as needed.

The word “revitalize” means that after the air stream has passed through the sorbent material of the present invention, some or all CO₂ is absorbed and O₂ is co-generated. A rebreathable air, a rebreathing air and a revitalized air are all defined as an air stream that has been revitalized by the sorbent material of the present invention, and they can be used interchangeably. However, a rebreathed air or a breathed air is a fouled air as described above with or without its excess carbon dioxide being reduced. That is, unless otherwise noted, the rebreathed air or breathed air might contain excess CO₂.

Further, in the present application, Fe(IV), Fe(VI), Fe(V), and a mixture thereof are referred to interchangeably as “ferrate,” and/or “ferrate compound.” The ferrate ion is referring interchangeably as Fe(IV), Fe(V) ion Fe(VI) ion, and/or a mixture thereof.

While not wishing to be bound by theory, it is presently believed that the ferrate ions drive the absorption of the CO₂ in the incoming air stream, replenish some of the oxygen used, and lower the humidity level of the foul gas. In this manner, the incoming air stream flows or diffuses through the sorbent composition/material to be freshened or revitalized to the level that is suitable for re-breathing. Lowering water content in the rebreathing air is not a critical feature for a CO₂ sorbent material; however, in some situations, such as underwater scuba diving, fire fighting, and confined living spaces, lowering the humidity level of the breathing air can provide more comfort to the user.

In addition, it is presently believed that the highly specific reactivity of the ferrate(VI) and other qualities of the ferrate compound provide a high CO₂ absorption capacity per unit volume of absorbent per volume of gas treated. That is, for the same volume of absorbent as that of a soda lime sorbent material and given the same gas flow rate and pressure of fouled breathing air, Fe(VI) absorbent material can last double the length of time for CO₂ absorption as that of the soda lime sorbent material. For example, it is calculated that a CO₂ sorbent material containing potassium ferrate(VI) is capable of absorbing CO₂ up to 44% of its volume. (It should be noted that CO₂ absorbent technologies or equipment are compared by volume not by weight.) If the ferrate sorbent material contact surface area for activating ferrate ions is optimized by using methods such as granulation to achieve lower pressure drop, the absorption yield of CO₂ can be increased relative to soda-lime canisters to about 90 vol/vol % to about 100 vol/vol %. Such optimization will adjust the phase change from one type of solid ferrate to a more freely contactable solid ferrate.

On the other hand, the highest possible CO₂ absorption yield by the CO₂ sorbent material based on soda lime is in the range of about 25% to about 35% of sorbent volume. For example, for Ascarite®, the popularly used CO₂ sorbent material brand used in laboratories and in industrial applications, the CO₂ absorption yield is only in the range of 20 to 30 vol/vol %. It is known that the soda lime sorbent material is limited by calcium carbonate coating or covering forming on the expanded solid particles of the sorbent material, which blocks or plugs the pores of the CO₂ sorbent material upon usage, preventing the remaining sorbent material trapped beneath from contacting the CO₂ of the incoming air stream.

Presently, ferrate is known to generate O₂ under acidic conditions. However, in the acidic conditions, the ferrate compound will not absorb any CO₂. At the acidic conditions (mostly below pH 6), CO₂ cannot remain dissolved in water as carbonate/bicarbonate ions to be absorbed by the ferrate compound; instead, it will escape into air as CO₂ gas. Without CO₂ being reduced in the rebreathed air, the mere generation of O₂ into a rebreathed air would not reduce the toxicity of the re-breathed air because the presence of excess CO₂ is toxic to humans. Excess CO₂ in the air would disable the blood proteins, leading to a rapid onset of illness and even death by changing pH of the environment for the proteins in the human body, beyond the scope within which the human proteins are able to withstand. Proteins in the human body, especially those in the blood cells, are very sensitive to pH changes in their environment. Typically, blood proteins can only withstand a few tenths of a pH unit change before the blood proteins become disabled, leading to the associated illness or death.

After the CO₂ is discharged by the body tissues, the discharged CO₂ gas must be released through the lung quickly. Otherwise, CO₂ would form carbonic acid/bicarbonate acid in the aqueous environment of the human body, which would then lower the pH of the blood in a human beyond the blood proteins' capability, disabling the blood proteins and causing the associated illness or death.

The air stream of an exhaled breath from a human typically contains about 40,000 ppm of CO₂. CO₂ from the exhaled breath can quickly increase the CO₂ level of a confined space, if the space does not have ventilation, such as a blocked mine, or a rebreather for underwater divers. If a human rebreathed in the air with excess CO₂ without having the CO₂ reduced or absorbed away, a huge amount of CO₂ gas would then enter into the human blood stream, react with the water in the blood, generate carbonic acid/bicarbonate acid, and lower the pH lethally for the blood protein. As a result, the human would quickly become sick or die, even if the rebreathed air is filled with oxygen gas.

The CO₂ absorption chemistry involving ferrate is believed to be represented as follows in equation (A):

4CO₂(g)+2M₂FeO₄(s)+3H₂O→MHCO₃(s)+3/2O₂(g)+2FeOOH(s) (A)

M represents one or more metal cations. That is, M can be any one or a blend of monovalent and/or divalent metal ions, which can be selected from a group consisting of an alkali metal ion, alkaline earth metal, a nonoxidizable transition metal ion, a group IIIA metal, a group IVA metal, a group VA metal, a lanthanide metal ion, and a mixture thereof. Unlimited examples of the metal cation are Li, Na, K, Rb, Cs, Mg, Ca, Sr, Ba, Sc, Y, La, other lanthanide, Zn, Cd, Al, Ga, In, Tl, Pb, Bi, or mixtures thereof. Most preferably, M in the ferrate compound is potassium ion.

The metal cations chosen will vary the solubility of the ferrate compound. For example, strontium and barium cations would produce ferrate compounds with a low solubility in water in the range of about 0.001 ppm to about 2000 ppm at a temperature in the range of about 0° C. to about 71° C. In the present invention, the ferrate compound with higher solubility and less volume is preferred, such as lithium ferrate, sodium ferrate, potassium ferrate, or a mixture thereof.

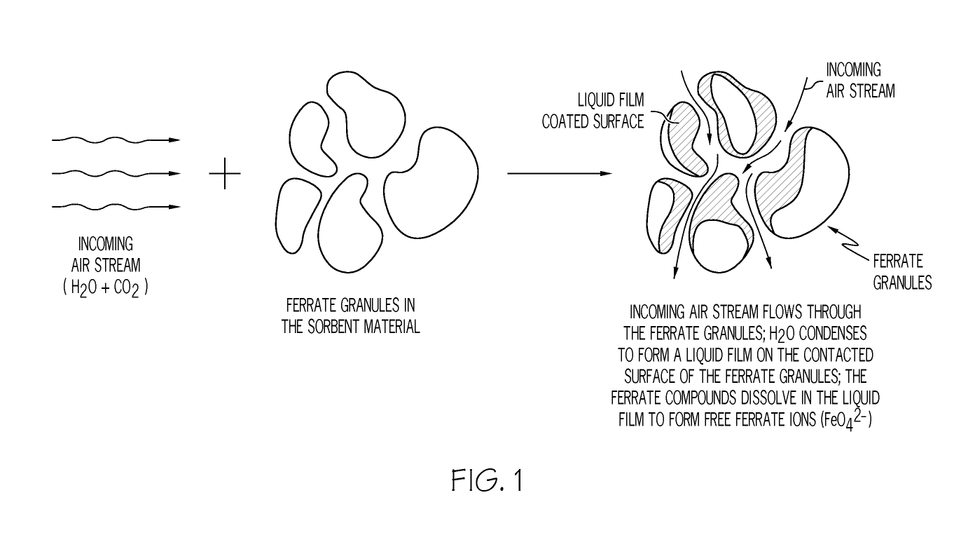

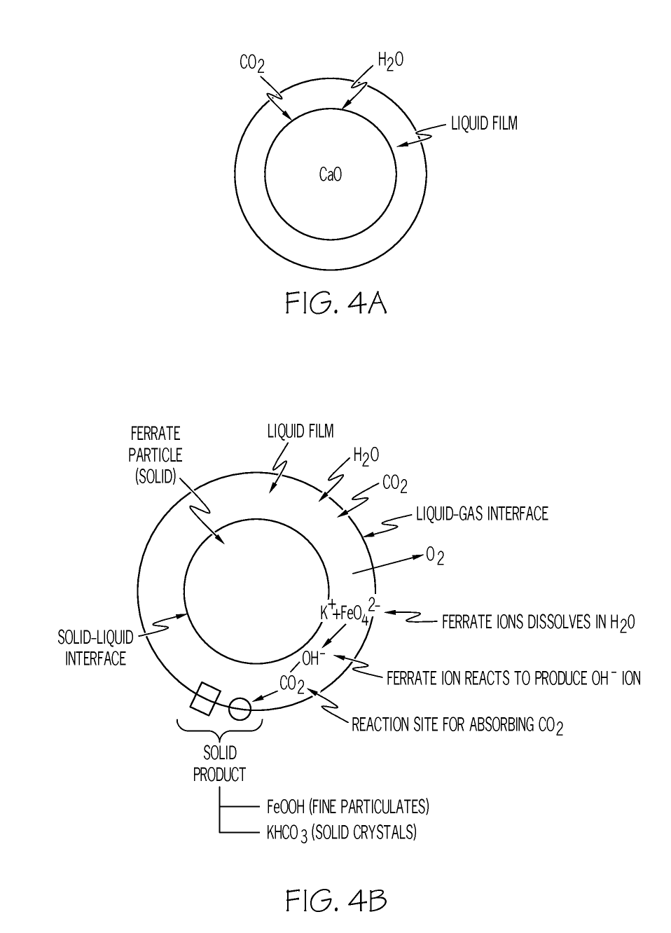

This above reaction (equation (A)) is believed to occur in several intermediate stages, shown below through reactions (1) to (3) (which are also illustrated in FIGS. 1 and 4B):

First, after an incoming air stream containing CO₂ and H₂O enters into the sorbent composition/material of the present invention, H₂O in the incoming air stream contacts parts of the surfaces of the sorbent particles and condenses to form a layer of liquid water film on the contacted surface. Two factors are believed to contribute the formation of the liquid water film layer: one is H₂O supply; the other is the temperature. Most of the time, the combination of the water supply and temperature are needed to be controlled or maintained to enable the formation of the liquid water film layer. In some cases, if the moisture in the incoming air is insufficient, part or all of the liquid film layer can come from (1) H₂O from the sorbent composition, preferably through the hygroscopic material, and/or (2) H₂O from one or more additional water sources close to the sorbent composition or material.

Typically, the ferrate compounds do not attract or absorb water by themselves, although they can react or dissolve in water. The human breath can contain lots of water vapor (moisture) along with CO₂. In a cold and very humid environment, such as during underwater diving, the moisture from the breath (or in the incoming air) would naturally condense on the sorbent material once it contacts the sorbent composition/material. In other words, the force from the breathing and/or one or more pumps drives the water vapor to the sorbent material, and the cold temperature then condenses the water vapor on the sorbent material, resulting in a liquid film layer.

The temperature suitable for the present invention is preferably about a dew point. The dew point is the temperature that the water vapor condenses into liquid water for a given portion of humid air and barometric pressure. Humidity and/or barometric pressure can influence the dew point. The humidity of the air can be increased by the additional water, such as through a water container, a humidifier, or any known devices that can provide H₂O. Preferably, such devices are small and light enough to avoid adding too much bulk and/or weight to the breathing system of the present invention using the ferrate sorbent composition/material. The pressure can be generated through a pump or other similar devices.

In some embodiments, a liquid water layer can be created and/or maintained on the sorbent composition/material at a temperature higher than the dew point. For example, the liquid water layer can be created by the hygroscopic material in the sorbent composition/material. The deliquescent hygroscopic material can absorb water and then liquidify upon exposure to additional water, and thus it provides a liquid layer without having to condense water at the dew point. Moreover, some hygroscopic polymers can provide water on their surface at a temperature higher than the dew point.

Nevertheless, the temperatures of the sorbent composition/material and/or the nearby environment need to be below the temperature in which most of the water would exist in its vapor form (the vapor temperature), such as 100° C. at a normal atmosphere. Below that vapor temperature, some excipients, such as hygroscopic material, can provide a liquid water film layer without the need for condensing H₂O. Above that vapor temperature, H₂O would escape as water vapor regardless of the hygroscopic materials. In some situations, such as fire rescue, temperatures are often above the vapor temperature, and as such, cooling equipment or agents are needed to keep the temperature below the vapor temperature.

The temperature needed for the formation of a liquid water layer on the sorbent composition/material can be achieved and/or maintained through the naturally cold environment, such as cold water in underwater diving. Alternatively, the suitable temperature can be controlled to enable the formation of the liquid water layer on the sorbent composition/material through these methods: (1) adding cooling agents or cooling components or equipment (collectively referred to as “cooling agent”) to the sorbent composition/material; and/or (2) increasing the surface area for the ferrate compounds in the sorbent composition/material through various formulations, such as granulation, coating on substrate, or other similar methods. Even in underwater, an environment that is typically cold, the water might be cold enough to cool the sorbent material/composition, either because the water might not be very cold or can even be slightly warm during a certain season or in a certain depth, and/or because at the local level, the exothermic nature of reactions for the CO₂ absorption and O₂ co-generation can create local heating regardless of the general cold environment. In other words, the cooling agents can cool the sorbent composition/material; and at the same time or alternatively, the ferrate compounds can be spread out in the sorbent composition/material so as to assist with the cooling, especially to dissipate the heating from any possible local heating from the exothermic reactions of CO₂ absorption and O₂ co-generation. Ideally, H₂O from the incoming air stream (or elsewhere) can be consistently and/or uniformly distributed through the entire sorbent composition/material to form water layers near the ferrate compounds for the dissolution of the ferrate compounds. Then the reaction rates (CO₂ absorption and O₂ co-generation) can be controlled so as to provide CO₂ absorption and O₂ co-generation as needed, either immediately and/or over an extended period, through methods known to one skilled in the art. Methods include coating the ferrate on porous substrates or glass beads, and/or embedding the ferrate compounds in solid solution or encapsulate the ferrate compounds (both of which are discussed in detail below or elsewhere in this application).

Preferably, the cooling agents act as intermediate agents to receive and discharge heat as needed, which can be immediately and/or continuously. The effectiveness of the cooling agent can be enhanced through the spreading-out of the ferrate compounds in the sorbent composition/material through various formulations. The purpose of the cooling agent is to reduce and/or control temperatures near the ferrate sorbent composition/material to be near or at the dew point to enable CO₂ absorption and O₂ co-generation. In other words, the cooling agent can dissipate or transfer heat either from the environment or from the heat generated from the exothermic reactions of the sorbent material during the CO₂ absorption and O₂ co-generation process. Local heating from the reaction process can be a problem when the ferrate compounds are packed into a crowded or packed space, preventing condensation of moisture on the ferrate, creating a barrier against any further moisture absorption by sorbent composition/material, and halting CO₂ absorption and O₂ co-generation. As such, the sorbent material can be used in both warm temperature and/or congested space so long as the temperature is not higher than the vapor temperature.

The cooling agents can be chemical compounds/materials, mechanical equipment components, or both. The chemical compounds/materials can be placed within the sorbent composition or material, such as phase changing materials or particulates. Suitable cooling agents, if they are chemical compounds, are basic and compatible with the ferrate compounds and any other ingredients in the sorbent composition/material. Alternatively, the cooling agent can be placed outside of or next to the sorbent composition/material as a heat exchanger, such as an aluminum or ceramic vessel containing the sorbent composition/material. Aluminum or ceramic vessels are preferably very light weight and thin to prevent them from taking up too much weight and space, which might make the breathing equipment containing sorbent composition/material to be too heavy or bulky for some of the desired applications.

In addition to or in combination with the temperature control, the additional water can be provided to the sorbent composition and/or material before and/or during the CO₂ absorption and O₂ co-generation. The additional water can provide both moisture (H₂O) and cooling to the sorbent composition/material. The additional water can come from (1) the hygroscopic material in the sorbent composition; (2) water in the form of liquid or vapor through one or more additional components; and/or (3) other similar mechanisms.

Preferably, the additional water comes from hygroscopic material in the sorbent composition/material. Hygroscopic material refers to one or more hygroscopic compounds, composition, and/or material. The hygroscopic materials suitable for the present invention are compatible with the ferrate compound. For the purpose of the present invention, the suitable hygroscopic material should provide H₂O for the liquid water layer needed for absorption of CO₂ and co-generation of O₂.

More preferably, the hygroscopic material is deliquescent. “Deliquescent hygroscopic materials” refer to substances (mostly salts) that have a strong affinity for moisture and will absorb relatively large amounts of water from the atmosphere if exposed to it, forming a liquid solution. Unlimited examples of suitable deliquescent hydroscopic materials include calcium chloride, magnesium chloride, zinc chloride, potassium carbonate, potassium phosphate, carnallite, ferric ammonium citrate, potassium hydroxide, sodium hydroxide, and a mixture thereof.

Alternatively, the hygroscopic material can carry moisture on their surface instead of absorbing moisture. Other polymers, such as polyethylene and polystyrene, do not normally adsorb much moisture, but are able to carry significant moisture on their surface when exposed to liquid water.

In some embodiments of the present invention, the hygroscopic material can be acidic, with a pH preferably not lower than 5; the acidity of the hygroscopic material is believed to be easily overcome by the ferrate compounds, which would naturally turn the surrounding liquid into an alkaline environment, which would then keep the ferrate from decomposition. Preferably, the hygroscopic material suitable for the present invention is not a pH buffer. A pH buffer is currently believed to interfere with the pH changes needed for the CO₂ absorption and O₂ co-generation by the ferrate sorbent composition/material. For example, initially, the sorbent composition is likely to have an alkaline pH due to the dissolution of the ferrate compound. Then, the dissolution of CO₂ in the liquid water reduces the pH from being highly basic to neutral or slightly basic (in some cases, slightly acidic). Soon, the reaction of ferrate with water to generate O₂ increases the pH to highly basic.

Preferably, the hygroscopic material has a neutral or basic pH. More preferably, the hygroscopic material has a slightly basic pH. However, if the hygroscopic material is strongly basic, such as KOH, only a small amount is preferably used, preferably in the range of about 1 wt % to about 5 wt % (based on the weight of the sorbent composition/material). If a large amount of the highly basic hygroscopic material is used, the sorbent composition/material would be subjected to a highly alkaline environment for an extended period, and it would take a long time for CO₂ to reduce the pH to the level that the ferrate compound can start to generate O₂. In a basic environment, the ferrate compound would not degrade or react even in the presence of available water layer. For the ferrate compounds to generate O₂ and ultimately remove CO₂ as a solid, the pH needs to be reduced to at least below 10, preferably below 9, and most preferably below 8. However, for CO₂ to dissolve or remain dissolved in water, the pH is preferably above 6, more preferably above 7. Otherwise, CO₂ would escape as a CO₂ gas in a lower pH regardless of how much liquid water is available to it.

More preferably, the hygroscopic material should have one or more common ions with the ferrate compounds. For example, if the ferrate compound is potassium ferrate, the hygroscopic material can be potassium nitrate or potassium hydroxide. The hygroscopic material and the ferrate compound both have the potassium metal ion, which is called the common ion. The addition of this type of hygroscopic material to the sorbent composition/material would create a common ion effect. Typically, the common ion effect causes a reduction in solubility of the salt, such as the ferrate compound. As such, such hygroscopic material can absorb and store H₂O while keeping part or all of the ferrate compound from being dissolved in the stored H₂O, and this status continues until additional water is introduced.

The hygroscopic material is preferably used to provide mostly water to the sorbent material initially and through the reaction process. Too much hygroscopic material would add significant amount of bulk (volume) and/or weight to the sorbent composition/material through its own weight and the weight of water absorbed, interfering with the practical use of the sorbet composition/material. Therefore, it is desirable that only a small amount of the hygroscopic material should be used as long as it provides a sufficient amount of coating or film consistently to all of the ferrate compounds. Sufficiency of the hygroscopic coating is determined by whether or not it can achieve its goal of attracting and/or providing H₂O to form a liquid water layer on the sorbent composition, preferably forming a liquid water layer on the ferrate compound.

The hygroscopic material can be added to the sorbent composition/material through methods known to one of ordinary skilled in the art. For example, a 3 wt % KOH can be combined slowly in the form of 50% KOH solution with the ferrate compound by milling the mixture with small glass beads in a container.

Furthermore, the additional water can also be provided through one or more water components (component comprising H₂O) next to or near the sorbent composition and/or material. This method of providing the additional water can be used with or without the hygroscopic material, which can be added to the sorbent composition and/or sorbent material. The “water component” in the present invention refers to anything that contains water. The water component can be H₂O from one or more hygroscopic materials in the substrate of the sorbent material. The water component can also be a crushable package of H₂O next to the ferrate sorbent composition. The crushable package can have a breakable wall, which can release H₂O upon need. H₂O from the package can be released as water vapor or moisture to the incoming air stream, or it can be released as liquid water directly onto the sorbent composition/material.

Then, some of the ferrate compounds near the liquid film dissolve in water at the solid-liquid interface between the solid ferrate particle and the liquid film, resulting in free ferrate ions immersing in the liquid film, as shown by intermediate reaction (1) (also see FIGS. 1 and 4B):

2M₂FeO₄+humidity→4M⁺+2FeO₄⁻²(liquid film) (1)

Then, the free ferrate ions in the liquid film on the sorbent surface react with water in the liquid film to generate O₂ gas and OH⁻ ions, as shown below by intermediate reaction (2):

4M⁺+2FeO₄⁻²+3H₂O→3/2O₂(gas)+4OH⁻+2FeOOH(s)+4M⁺ (2)

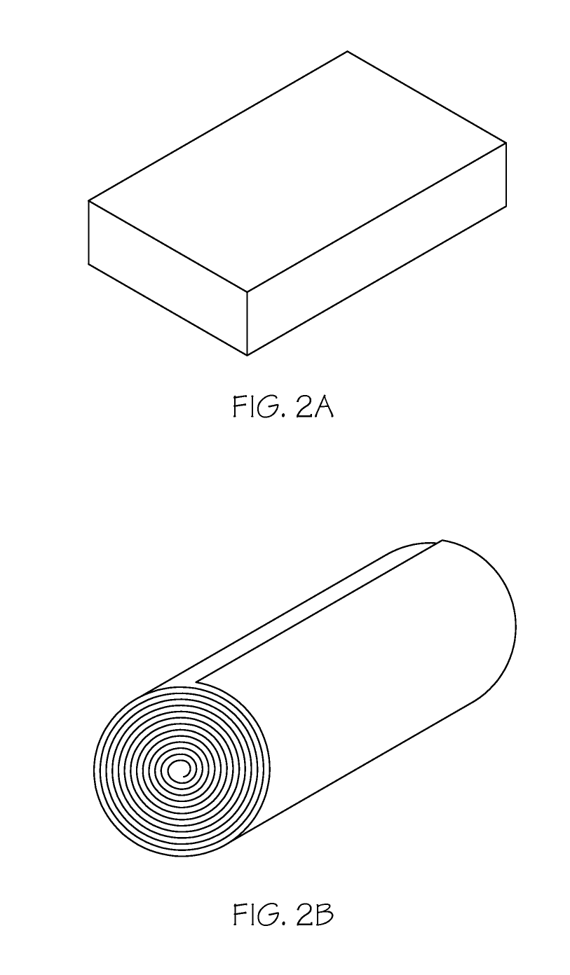

Immediately following the formation of OH⁻ ions, the CO₂ gas is absorbed from the incoming air stream to form a second solid phase of bicarbonate on the outer surface of the liquid film (also called the liquid-gas interface between the liquid film and the incoming air stream) (see FIG. 2B). The bicarbonate then neutralizes the OH⁻ ions, as shown by intermediate reaction (3) below. The absorption of CO₂ gas is further illustrated and explained later.

4M⁺+4OH⁻+4CO₂→4MHCO₃(solid) (3)

In the intermediate reaction (2), while not wishing to be bound by theory, it is currently believed that the ferrate ion might have reacted to produce O²⁻ possibly because the ferrate ion (FeO₄²⁻) is an oxo-metal ion complex. The theoretical O²⁻ ion would then immediately react with the water molecule to produce the hydroxide ion (OH⁻). This O²⁻ is extremely basic and does not exist by itself in water. It is used to explain how the ferrate ion might have reacted with a water molecule to produce the hydroxide ion as follows:

O²⁻+H₂O→2OH⁻

The key step is the intermediate reaction (3), in which the intermediate reaction product, M⁺OH⁻, reacts with CO₂ to produce a neutral MHCO₃. This intermediate step is not known currently, although Fe(VI) is known to produce O₂ and to increase the pH of the solution to be strongly basic by forming lots of hydroxide ions. The CO₂ gas on the left side of the reaction (3) is already present in the incoming air stream entering the sorbent material. The O₂ gas on the right side of the reaction (2) along with the removal of the CO₂ and moisture (reactions (2) and (3)) together represent the formation of fresh (revitalized) breathing air that then can be allowed to exit the sorbent material.

As such, while not wishing to be bound by theory, it is currently believed that the triggers for the above equations are CO₂ and moisture near the sorbent material. The dual triggers provide a semi-self regulation of the CO₂ absorption rate to meet the demand of the person using the equipment containing the CO₂ sorbent material of the present invention, which also regulate the O₂ replenishment and humidity reduction rates. This semi-self-control aspect of the invention is described in more detail below. In other words, the control of the ferrate action in the sorbent material is provided by the CO₂ gas flow rate and the condensation rate of the moisture vapor from the incoming moist air stream.

First, as shown by FIG. 1, the vapor moisture in the incoming air stream near the sorbent material cools sufficiently to condense into a film of water (liquid film) on the surfaces of ferrate granules in the sorbent material composition (see FIGS. 1 and 4b). As shown by FIG. 4B, there are two interfaces for two surfaces of the liquid film. One interface is a solid-liquid interface between the solid surface of the ferrate particle and the inner liquid surface of the liquid film that is in contact with the solid surface of the ferrate particle. The other interface is a liquid-gas interface between the outer liquid surface of the liquid film and the incoming air stream, where the outer liquid surface is in contact with the incoming air stream. The water in the surface liquid (H₂O film) dissolves a small amount of ferrate compounds/compositions on the surface of the ferrate granule at or near the solid-liquid interface, and the dissolved ferrate compounds dissociate into metal cations and ferrate anions as shown in equations (a) and (b) below.

Vapor moisture+cooling condensate H₂O film on the surface of the sorbent material. (a)

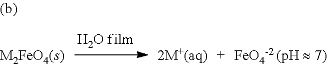

The metal cations and ferrate ions migrate in the liquid film to the liquid-gas interface between the liquid film and the incoming air stream, where the ferrate ions interact or react with the dissolved aqueous CO₂ as explained below (also see FIG. 4B).

At the same time as the ferrate compound is dissolving in the liquid film at the solid-liquid interface, CO₂(g) in the incoming air stream dissolves in the water to form aqueous CO₂ at the liquid-gas interface of the liquid film (see FIG. 4B). The aqueous CO₂ then reacts with the water at or near the liquid-gas interface of the liquid film to generate carbonate acid (equation (c)), which is followed by production of carbonate ion and hydrogen ion (equation (d)). As the result, CO₂ generates a mild acidity through equations (c) and (d), attempting to drive the pH of the surface film to about 4-6 from the pH of about 7 (see equation (b)).

4CO₂(aq)+4H₂O⇄4H₂CO₃ (c)

Followed by:

4H₂CO₃⇄4HCO₃⁻+4H⁺(pKa of HCO₃⁻ is about 6.3, which attempts to drive pH down to ≈4-6) (d)

The presence of the H⁺ ions from equation (d), even in a trace amount, attracts the ferrate ions and causes the protonation of the ferrate ions from equation (b), FeO₄⁻². The reaction of H⁺ ions and the ferrate ions results in a highly reactive intermediate product, HFeO₄⁻, as shown in equation (e):

FeO₄⁻²+H⁺⇄HFeO₄⁻(highly reactive oxidant, pKₐ=7.3) (e)

The formation of HFeO₄⁻ then triggers a rapid (essentially instant) oxidation of water to form oxygen gas (O₂), strong base (OH⁻), and iron(III) solid product (FeOOH(s)), as follows in equation (f), which attempt to drive the pH to about 10-12:

2HFeO₄⁻+H₂O→3/2O₂(g)+2FeOOH(s)+2OH⁻(pH≈10-12) (f)

The resulting high pH instantly stops any further reaction from equation (f) because HFeO₄⁻ is no longer present at this high pH as its pKₐ value is only about 7.3.

However, when more CO₂ gas enters into the atmosphere near the sorbent material, preferably from the next exhaled breath of the user, the CO₂ gas is rapidly and efficiently absorbed by the highly basic liquid film on the surface of the sorbent material from reaction (f) to produce first carbonic acid (H₂CO₃) as shown by equation (c). The first carbonic acid then immediately reacts with the hydroxide ions from equation (f) to form the carbonate ions (HCO₃⁻) as follows in equation (g):

4OH⁻+4H₂CO₃→4HCO₃⁻+4H₂O(pH buffered at ≈8-8.5) (g)

Further, as the reaction based on equation (g) proceeds, the bicarbonate ions product (4HCO₃⁻) from equation (g) and/or equation (d) becomes highly concentrated in the thin liquid film on the surface of the sorbent material. As the result, the bicarbonate ion product crystallizes with the metal cation(s) originally introduced with the ferrate ion to form a separate bicarbonate (and even carbonate) crystal solid(s), which is shown in equation (h). It is interesting to note that as shown by FIG. 4B, the bicarbonate or carbonate crystals are formed on the liquid-gas interface of the liquid film

4M⁺+4HCO₃⁻→4MHCO₃(s)(solid crystals) (h)

Therefore, in addition to CO₂ and H₂O, the drivers for the above equations (a) through (h) include the relative pH level in the aqueous film on the surface of the ferrate sorbent material.

Equations (a) and (b) show that the presence of the moisture (H₂O) in the incoming air stream allows parts or all of the ferrate compound to dissolve to produce free ferrate ions (FeO₄²⁻) in the solution present in the aqueous film on the surface of the ferrate sorbent material (see FIG. 1). The dissolved ferrate ions have a pH of about neutral because pKₐ of HFeO₄⁻ from equation (e) is about 7.3. Then in the equation (f), the free ferrate ions react with water molecules to produce hydroxide ions, which increase the pH to about 10-12. Therefore, the reactants on the left side of the equation (f) have a pH of about 7.3, while the products on the right side of the equation (e), i.e. OH— and FeOOH products, have a pH of about 10 and higher.

At the same time, the equation (c) shows that carbon dioxide dissolves in or reacts with water to produce carbonic acid (H₂CO₃), which resulted in a pH of about 4-6 due to the reaction from equation (d). This carbonic acid then reacts with the hydroxide ions resulted from equation (e) through equation (f) to produce the bicarbonate ions and water, which increases the final reactants' pH on the right side of equation (g) to a mildly basic pH, about 8 (range in 7-9). The absorption of the CO₂ gas then drives the ferrate ions to continue to react with the water molecules to replace the absorbed hydroxide ions (equation (f)).

Before going further, it is important to note that carbon dioxide is soluble in water, in which it spontaneously interconverts between CO₂(aq) and H₂CO₃ (carbonic acid). The relative concentrations of CO₂, H₂CO₃, and the deprotonated forms, HCO₃⁻ (bicarbonate) and CO₃⁻² (carbonate), depend on the pH of the solution in a predictable manner. In a neutral or slightly alkaline condition (10>pH>6.5), the bicarbonate ion form predominates (>50%), while in a very alkaline condition (pH>10.4) the predominant (>50%) form is carbonate ion. As such, any further reduction in pH (to <6, sometimes to <7) through the addition of any acid, including CO₂ gas, would keep carbon dioxide in its CO₂ gas form; while any increase in pH, which can be caused by free ferrate ion, would drive carbon dioxide to react with the basic component to produce bicarbonate ion or carbonate ion.

The ratio between ferrate and CO₂ would determine which deprotonated forms of the carbonate ion, bicarbonate ion or carbonate ion, is produced from the reaction between CO₂ and the basic component (OH⁻). In the present invention, initially, the product of CO₂ is carbonate ion because the ferrate absorbent is in excess and the aqueous film can be very basic. Then, the product of CO₂ shifts to bicarbonate ion as more CO₂ is absorbed and the aqueous film becomes less basic.

In other words, the liquid film on the ferrate particle surfaces from the vapor moisture enables the ferrate compounds in the sorbent material to dissolve and react to produce the initial hydroxide ions and oxygen gas at about pH of 10 or more. At the same time, the CO₂ gas is being absorbed by the same liquid film to form dissolved aqueous CO₂ and its associated carbonic acid. Then, almost instantaneously, the higher pH (slightly alkaline condition) drives carbon dioxide to produce carbonic acid, which has a pH of about 4-6. Carbonic acid then reacts with the hydroxide ions, which reduces the pH from about 10 to about 8. The lowering of the pH by carbon dioxide then would drive the equation (f) to proceed to the right side to produce more hydroxide ions in order to increase pH.

Therefore, while not wishing to be bound by theory, it is presently believed that the absorption of carbon dioxide by the ferrate compound would only occur in a slightly alkaline condition, in which pH is preferably in a range of about 6 to about 10, more preferably in a range of about 6.5 or 6 to about 9, and most preferably in a range of about 7 to about 8. If any acid is added, carbon dioxide would not convert into carbonic acid or bicarbonate acid. In fact, if any acid is added, the ferrate compound would release or produce carbon dioxide gas as disclosed by other references, although this ferrate compound would still produce oxygen gas. For example, in US patent application publication 2009/0061267 and PCT published application WO/2007/027876 to Monzyk et al., acid was needed in the form of H⁺ to drive the production of O₂ from ferrate.

Further, the above equations showed a solid to solid conversion process, in which the solid particles of ferrate compound are converted to solid particles of FeOOH and MHCO₃. From equation (a) through equation (h), solid ferrate particles in the sorbent material are gradually transformed into nontoxic, environmentally neutral moist solid particles of FeOOH and crystals of MHCO₃, which can be used or disposed of as nonhazardous materials. Uses for the resulting FeOOH and MHCO₃ materials typically include iron feeds to steel production mills, materials for the neutralization of waste acids, iron micronutrient fertilizers additive, and the like.

More importantly, because the combination of carbon dioxide and H₂O drives the equation (A) by using the ferrate compound to absorb carbon dioxide and to co-generate oxygen, the CO₂ sorbent material composed of the ferrate particles would have an extended CO₂ absorption capability in comparison to that of the existing CO₂ sorbent materials composed of soda lime.

The existing sorbent materials use “soda lime” (lime coated with a small amount of sodium hydroxide). Lime is very insoluble in a liquid solution or in water. Upon exposure to CO₂ and water, a layer of liquid film is first condensed on the surface of the lime (CaO) particle. Then, in its solid particulate form, lime (CaO) absorbs CO₂ and H₂O from the liquid film through the action of the sodium hydroxide coating. After H₂O absorption, the lime (CaO) particle would swell or expand because CaO is insoluble in water, while the liquid film would either disappear or be dramatically reduced to be almost negligible. So, after the CO₂ absorption, the resulting calcium carbonate is not deposited on the outer surface of the liquid film, instead it is deposited on the top of the expanded solid soda lime sorbent material. Over time, the resulting calcium carbonate particles would bury or coat the entirety of the remaining lime material. Therefore, the expansion of the lime particle along with the solid calcium carbonate coating would block the remaining lime from being able to absorb CO₂ after a period of time. As such, soda lime sorbent material loses its capacity to absorb CO₂ over time.

In the present invention, the sorbent material would only continue its reaction with water to produce hydroxide ions when carbon dioxide and H₂O are present. Without carbon dioxide, the ferrate compound or ion would not continue its reaction with water or at least reduce its rate of reaction with water. Without such continued reaction, no solid particles of FeOOH and MHCO₃ would be produced.

Further, while not wishing to be bound by theory, it is presently believed that the solid products, FeOOH and MHCO₃, on the surface of the ferrate particles would not block the ferrate sorbent material from further reactions to absorb CO₂ and water. First, as shown by FIG. 4B, unlike lime (CaO) particles, the ferrate particles dissolve in water to form ferrate and metal ions at the solid-liquid interface of the liquid film. As such, the ferrate particles would slowly reduce in size as they dissolve to react with CO₂ and water. The ferrate and metal ions would migrate to the liquid-sold surface of the liquid film to react with water and CO₂ to form the resulting solid products, FeOOH particles and MHCO₃ crystals (see FIG. 4B). The solid products might fall off the liquid film or be easily dislodged from the liquid film surface. Second, the co-generation of O₂ gas bubbles on the ferrate surface would also dislodge the solid product to ensure that there are surface spaces on the ferrate particles available for absorption of CO₂ and water.

More importantly, the ferrate sorbent compositions have a higher CO₂ absorption capacity because the ferrate sorbent compositions increase their volumes much slower than that of the soda-lime sorbent materials. As explained above, the ferrate composition absorbs CO₂ while co-generates O₂. While not wishing to be bound by theory, it is currently believed that the increase in volume caused by the CO₂ absorption is somewhat offset by the decrease in volume caused by the release of the O₂ gas. On the other hand, soda-lime sorbent materials do not produce any oxygen during their CO₂ absorption process. Therefore, soda-lime sorbent material would only increase its volume during CO₂ absorption.

In conclusion, while not wishing to be bound by theory, it is presently believed that for the above reasons of (1) CO₂ being the driver, (2) ferrate compound being able to dissolve in water and (3) co-generation of O₂ gas, the sorbent material with ferrate can last much longer than the other sorbent material of soda-lime and provide a greater CO₂ absorption capacity ratio (gas volume/sorbent volume).

According to some embodiments of the present invention, as explained above, a sorbent composition comprising the ferrate compound (also called ferrate sorbent composition) is suitable to revitalize a fouled or breathed air stream by absorbing CO₂ and co-generating O₂. While not wishing to be bound by theory, it is presently believed that the reactions associated with CO₂ absorption and O₂ generation release certain amount of heat. The exothermic nature of the reactions can create some local heating, especially when the ferrate compounds are congested in a confined space. The local heating can contribute to the formation of one or more possible barriers against further absorption of CO₂ and H₂O by the ferrate compounds in the interior of the sorbent composition/material. In other words, this local heating would dry out the moisture in the surrounding ferrate particles, preventing further condensation of moisture on the ferrate within “the ferrate clumps,” and creating a barrier against absorption of moisture and/or CO₂. In other words, after the initial reaction which absorbs CO₂ and co-generates O₂, most of the ferrate particles would be blocked from accessing CO₂ and/or moisture. Without accessing either CO₂ and/or moisture, the ferrate particles would not be able to absorb CO₂ and co-generate O₂.

Therefore, it is critical to prevent local heating or control temperature to enable condensation of moisture on the sorbent composition. This can be accomplished by spreading out the ferrate compounds in the sorbent composition/material, and by the addition of cooling agents. The ferrate compounds can be spread out in the sorbent composition/material by methods such as forming the ferrate into granules, extrudates, spheres, disk, briquettes, pellet, prill, encapsulate, microsphere, solid solution, or a mixture thereof. These forms of ferrates prevent or reduce the possibilities of ferrates particles clumping together, and/or reduce the heat from the ferrate reaction. Such forms of the sorbent composition can typically be achieved through known granulation methods, extrusion processes, pelleting machines, prilling procedures, encapsulation processes, or a combination thereof. During these processes, sometimes it is preferred that one or more compatible and suitable excipients are added, such as suitable binders. For example, binders might be needed to form ferrate pellets, disks, prill or granules.

In a further embodiment, the sorbent material/composition provides for ferrate compounds in the form of granules, and the ferrate granules are coated with one or more hygroscopic materials mentioned above.

The temperature can also be controlled and/or maintained by the addition of cooling agents. The cooling agents can absorb and dissipate the heat from the general environment and from any possible local heating resulting from the exothermic reactions of the sorbent composition/material. The effectiveness of the cooling agents can be enhanced by the formulation efforts in spreading out the ferrate compound in the sorbent composition/material.

The temperature, water content, and CO₂ level can be controlled in the present invention through additional H₂O components (hygroscopic materials or other devices), cooling agents, and various formulations so as to control the rate of CO₂ absorption and O₂ co-generation by the sorbent composition/material.

In some embodiments of the present invention, a hygroscopic material can be added to the Fe(VI) sorbent composition. The hygroscopic material has the capability to attract or pull moisture in a low moisture environment to provide a source of water (H₂O) to react with Fe(VI) to absorb CO₂. Further, even in a humid environment, the addition of the hygroscopic material can be used to control the access of moisture to the Fe(VI) material in the sorbent. Preferably, the hygroscopic material is coated on the surface of the ferrate sorbent composition particles.

Suitable hygroscopic materials are compatible with the ferrate compound. The characteristics of the preferred hygroscopic materials are described in detail above in the application. Typical examples of the hygroscopic material suitable for the present invention include KOH, NaOH, K₃PO₄, CaCl₂, sodium, silicate, potassium silicate, and a mixture thereof.

In some preferred embodiments of the present invention, the sorbent material includes one or more cooling agents. The preferred cooling agents are described above in the present application. The cooling agents can be used in combination with the hygroscopic material and/or the optional H₂O component (also called “the additional H₂O component”) to assist in the formation of the liquid water layer on the sorbent composition/material as illustrated in FIGS. 1 and 4B. The hygroscopic material and/or the optional H₂O component can provide H₂O, while the cooling agents can ensure that the provided H₂O condenses or forms into a liquid water layer to be used in CO₂ absorption and O₂ co-generation.

Some broad embodiments of the present invention provides for a sorbent material suitable for removal of CO₂ and co-generation of O₂ include one or more ferrate sorbent compositions described above. Preferably, in the sorbent material of the present invention, the sorbent compositions are embedded in one or more fibers. The fiber can also include one or more hygroscopic materials to attract and/or provide more H₂O for the sorbent composition.

Preferably, the ferrate fiber can be produced by dispersing suitable ferrate formulations into one or more suitable nonaqueous polymers to produce the ferrate fibers. The suitable ferrate formulations are described elsewhere in the application, such as ferrate granules, solid solutions, encapsulates, etc. For the purpose of the ferrate fiber formation, the “nonaqueous polymer” is defined in this application as a polymer containing very little water, preferably containing no more than 3 wt % water. The nonaqueous polymer, in the context of this application, can dissolve in an aqueous solvent, or in a nonaqueous solvent, or both.

Preferably, the suitable or compatible nonaqueous polymer can be, but is not limited to, epoxy resin, alkyd, polyester, polyurethane, polyolefin, polyamide, polysulfide, polythioether, phenolic polyether, polyurethane, polyvinyl, rosins, polyesters, silicones, siloxanes, perfluorinated resin, other fluorinated resins, polytetrafluoroethylene (Teflon®), polyvinylidene difluoride, nylons and other polyamides, copolymers thereof, blends, or mixtures thereof. Some of the polymers may be somewhat hygroscopic. The hygroscopic polymers can absorb and retain a certain amount of water to prevent the moisture in the air from reaching the ferrate to prematurely decompose the ferrate ions. Hydroscopic polymers are nylons, other polyamides, polyurethanes, polyvinylalcohols, polyethers, cellulosics, silicones, and the like.

The ferrate fiber might also contain one or more nonaqueous solvents, and/or excipients, preferably nonaqueous excipients. The ferrate compound is preferably present in a concentration that does not interfere with the integrity of the resulting ferrate fiber. The fiber can be produced by conventional extrusion methods.

Alternatively, the fiber can be made using electric field effect technology. Electric Field Effect Technology (called “EFET” herein thereafter) includes electrospraying, electrohydrodynamic spraying (EHD), electric field spraying, electro-spinning, spray technology as exemplified by patents such as U.S. Pat. No. 6,252,129 to Coffee, U.S. Pat. Pub. No. 2009/0104269 to Graham et al., U.S. Pat. Pub. No. 2008/0259519 to Cowan et al., U.S. Pat. Pub. No. 2006/0194699 to Moucharafieh et al. (the contents of these patent and published patent application are hereby incorporated by their entirety), and the like. Further, “EFET” can be used interchangeably with “EHD.”

EFET embodies the process of utilizing an electric field to charge and subsequently extrude aerosol particles of microstructures, such as fibers, films or nano/microparticles etc., from a bulk liquid formulation. In the present invention, EFET is used to produce fibers of ferrate compounds. The size of the microstructures can be adjusted from fractions of a micron to hundreds of microns, depending on the specific application. Because the microstructures from EFET are electrically charged when they are formed, additional features may be leveraged, such as directivity of the microstructure through electrical means, and interactions among the generated microstructures to cause secondary formations, such as fiber mats. Distinctive advantages of EFET include its flexibility to produce a variety of structures, consistency of performance, and gentle handling of delicate materials, such as the highly oxidative ferrate(VI) compounds.

The EFET method for producing the ferrate fiber is preferred because the fiber size can be generally produced within a tight distribution range. The fiber is also produced such that the solvent for the ferrate can be evaporated or flashed off quickly. Further, the resulting ferrate fiber can also be coated or encapsulated in one continuous step using two or more nozzles via EFET.

In the ferrate fibers, the ferrate compounds in the sorbent composition are spread out more evenly, allowing for more even and/or immediate access to H₂O and CO₂, reducing local heating with or without the inclusion of cooling agents. The fiber formulation can be adjusted to provide more porosity, which would enhance the access of H₂O and reduction of local heating. The ferrate fiber can also release the ferrate ions in a controlled fashion so that the sorbent composition/material can have a capacity for immediate CO₂ absorption and O₂ co-generation, and/or can also have the capacity for CO₂ absorption and O₂ co-generation over an extended period of time, such as hours, days, or weeks.

The controlled release of the ferrate in the fiber and/or any other sorbent composition/material described above can be achieved through five factors: (1) the solubility of the ferrate compound or formulation; (2) the hygroscopicity of the fiber and/or the ferrate formulation; (3) pH control; (4) physical/mechanical abrasion exposing the embedded ferrate compound or formulation; and (5) the porosity of the fiber. These five factors are inter-related. For example, the hygroscopicity of the ferrate formulation can change the porosity of the fiber, and pH variation can change the solubility of the ferrate.

Specifically, the higher the solubility of the ferrate, the more free ferrate ions can be released upon exposure to the moisture. The higher the hygroscopicity of the ferrate formulation and/or the ferrate fiber, the more likely the ferrate crystals are exposed to the moisture in the fiber. The more porous the ferrate fiber is, the more likely the ferrate can be exposed to the moisture/water. The lower the pH of the ferrate/ferrate formulation, the higher the solubility and reactivity of the ferrate. In addition, the higher the hygroscopicity of the ferrate fiber, the more likely that the fiber might swell upon absorption of moisture to become more porous, which in turn, might provide more paths for the moisture to reach the ferrate, or for the freed ferrate to migrate to the moisture. As a result, the ferrate is discharged from the fiber (or brushes/pads/filters made of the ferrate fiber) to react with the surface/environment to be cleaned. Of course, the more the fiber is physically brushed or rubbed, the more likely the embedded ferrate crystals are exposed to moistures and/or contaminants.

Other factors include temperature, the shapes of the ferrate crystals, the aspect ratios of the ferrate crystals, the positioning of the ferrate crystals inside the fiber, which can also impact one or more of the above five factors. For example, the shape of the ferrate crystals can influence the solubility of the ferrate. Further, the shapes of the ferrate crystals might be used to control the porosity of the ferrate fiber or other ferrate sorbent composition material. In some embodiments, for purposes of having a faster or more immediate release of ferrate, the ferrate crystals are preferably embedded throughout the fiber and are placed so that the ferrate crystals are in physical contact with each other. For example, if a ferrate crystal A is in physical touch with a ferrate crystal B. After the ferrate crystal A is exposed to moisture, leached out, and reacted with the target microbes/chemical/contaminant(s), it leaves an empty space/pore, which then enables the moisture to reach the ferrate crystal B right next to the reacted ferrate crystal A space to release the ferrate ion from the ferrate crystal B. In other words, the shape, length, and the positioning of the ferrate crystals can create or increase the porosity of the fiber for the moisture to reach the ferrate crystals at a faster rate, delivering more free ferrate ions per any given volume of the ferrate fiber and/or other ferrate sorbent composition/material.

The greater the aspect ratio of a ferrate crystal, the more likely these ferrate crystals will touch each other physically at a lower loading percentage in the fiber. As such, the aspect ratio of a ferrate crystal can also control the rate of the release of the ferrate ion in the fiber. In some cases, the aspect ratio of a ferrate crystal is determined by its oxidative state. For example, sodium ferrate(V) compounds are usually in the shape of a long needle with a high aspect ratio, while potassium ferrate(VI) compounds have a more platelet or rhombic shape with a lower aspect ratio when compared to the ferrate(V) compound. Similarly, barium ferrate(VI) and calcium ferrate(VI) have small aspect ratios near 1 and a very small particle size. On the other hand, barium ferrate(VI) and strontium ferrate(VI) have a very high surface area volume unit of crystal. The long needle shape of the ferrate(V) compounds have a longer reach, up to at least 100 microns, which enables the ferrate(V) compound crystals to be in physical touch of each other at a lower loading volume percentage of the ferrate. Therefore, if a faster release of the ferrate ion is desired, the sodium ferrate(V) compound can be used instead of the potassium ferrate(VI) compound. In other cases, the ferrate(V) compounds can be used in conjunction with the ferrate(VI) compound to obtain variable rates of release of the ferrate ions. Barium ferrate(VI) and barium ferrate(V) have very low solubility so they can be used as the slow release ferrate compounds. In addition, using solid solution crystals of the ferrate and compatible ions, for example solid solutions of potassium ferrate and potassium sulfate, is another means to reduce the ferrate release rate where only very small amounts (e.g. 0.1-11 ppm) would be released over an extended period of time (e.g. in air filtration or water purification at the point of use, and the like).

All of the above factors discussed above are dependent upon the solubility of the ferrate compound to release the ferrate ion in controlling the reactivity of the ferrate. In the present invention, the solubility of the ferrate can first be controlled by the metal cation of the ferrate compound. The preferred metal ion for achieving the slower release of the ferrate ion from the ferrate compound is alkaline earth metal ion, such as strontium or barium. Such alkaline earth metal ions stabilize ferrate anions through forming salts of low solubility in both water and organic phase and enable them to exist in a very rare high oxidative state of Fe(IV), Fe(V), or Fe(VI). Specifically, alkaline earth metal ions, along with other metal ions mentioned above, can produce ferrate compounds with a low solubility in water in the range of about 0.001 ppm to about 2000 ppm at a temperature in the range of about 0° C. to at least 71° C., and sometimes to about 100° C.

On the other hand, the alkali metal ions form ferrate salts of relatively higher solubility in aqueous phases (as would be present in aqueous scrubbing) and moisture films (as would be present in air filters), while at the same time, the alkali metal ions enable the resulting iron salt to exist in a high oxidative state of Fe(V) or Fe(VI). The ferrate compounds with higher solubility can be used for immediate release of the ferrate ion, while the ferrate compounds of lower solubility can be used to release the free ferrate ions over time. Further, mixtures of ferrate compounds of different solubility can be co-dispersed/co-mixed inside of the fiber to achieve both immediate and extended release of the free ferrate ions upon use. This variable controlled release of the ferrate ions is very useful for both brushing action and the filtering uses.

The solubility of the ferrate compound can also be controlled by encapsulation and by placing the ferrate in a solid solution, such as ferrate doped potassium sulfate or potassium chromate(VI). The ferrate compound with a higher solubility can be encapsulated to control and/or reduce the release rate of the free ferrate ions. The encapsulation can be porous, allowing certain amount of moisture to permeate through to the ferrate compound to release the ferrate ions in a slower fashion. Such porous encapsulation can be accomplished by encapsulating the ferrate into a zeolite, aluminate, zircoaluminate, and the like. The encapsulation can also be nonporous, having little or essentially no permeability to moisture, liquid or vapor. This type of encapsulation can be done by encapsulating the ferrate with silica or potassium orthophosphate, or overgrowing the ferrate crystal with potassium sulfate. The nonporous encapsulation can enhance the stability of the ferrate compound of any solubility, especially that of higher solubility, to enable the ferrate to be compatible with other components of the ferrate formulation and to be compatible with the polymer in the fiber.

The nonporous encapsulation of the ferrate preferably has a hydrophobic coating or wall composed of hydrophobic excipients or materials. Inside of the microcapsules, one or more hygroscopic compounds or solvents can be included, in which the ferrate is substantially not soluble. The hygroscopic compounds absorb moistures inside themselves and away from the ferrate. This type of encapsulation is similar to a sealed chamber containing desiccants, in which the desiccants are hygroscopic and absorb the moisture away from the environment in the chamber, and thus keeping the moisture low in the sealed chamber. The encapsulation process also helps control the rate of ferrate release/reactivity in the scrubbing or filtering application.

Alternatively, or in combination with the encapsulation, ferrate ions of the ferrate compounds can be incorporated into solid solution crystals of low solubility with other compatible ions. The solid solution crystals can be made by the process of diffusion and/or absorption from aqueous solutions, sprays with tumbling, co-precipitation/co-crystallization, and the other acceptable techniques. Suitable compatible ions can include, but are not limited to, neutral or pH basic clays, minerals, low soluble salts, talcs, glass fibers (pH adjusted), silicates, inerts such as gypsum, sodium sulfate, and the like. Such formulated solids can reduce the rate of release of free ferrate ions in a controlled fashion because the bulk solid is very slow to dissolve, slow to leach in thin adsorbed moisture films, or it can be substantially insoluble. For enhanced control a selected amount of ferrate ions can be embedded in solid solution crystals through crystallization or ion exchange processes already known in the art. After incorporating the solid solution or formulation of the ferrate compound into the fiber, the solid solution crystals can act as filler carrier salts in carrying the ferrate ions in the fiber. As a carrier, the solid solution can facilitate the even spreading of the ferrate ion in the fiber even when there is a very a low concentration of the ferrate in the fiber. As such, the solid solution method can control the rate of release of ferrate ions in the fiber to perform cleaning/disinfecting functions; while at the same time, it can prevent spontaneous premature/useless decomposition of the ferrate ions.

The compatible ion can include, but is not limited to, a sulfate ion, a chromate ion, a silicate ion, an aluminate ion, an orthophosphate ion, a borate ion, a carbonate ion, a titanate ion, a zirconate ion, a manganate ion, a molybdate ion, or a mixture thereof.

According to some embodiments of the present invention, the sorbent material includes one or more ferrate sorbent compositions (also called the sorbent composition) which are joined with (incorporated into or onto) a substrate to form a sorbent layer. The substrate suitable for the present invention typically includes one or more matts (or mats), screens, beads, porous materials (paper, fabric or plastic), perforated plastic, perforated and corrugated plastic, woven or non-woven fabric, or mixtures thereof. Preferably, the substrate can be formed of any porous material compatible with the ferrate sorbent composition. Substrate can include one or more hygroscopic materials described above in the application. The hygroscopic materials can assist in attracting water in the air stream and/or providing additional water to the sorbent composition/material. The hygroscopic materials can also achieve some cooling effect through the absorption of water, which can also reduce local heating effect of the CO₂ absorption and O₂ co-generation by the sorbent composition/material of the present invention.

Preferably, one or more sorbent compositions described above can be coated on at least one substrate. The coating on the substrate will provide more surface area to spread out the sorbent composition, reducing or eliminating local heating, and allowing H₂O easier access to sorbent compositions. The coating can be accomplished by any known conventional coating methods.

Preferably, the sorbent layer can be compressed into a sheet or formed into a spiral as shown by FIGS. 2A and 2B. Further, the sheet form of the sorbent material can form a stack of sorbent sheets as a sorbent unit in a sorbent equipment. The shape of the sorbent sheet can be triangles, squares, rectangles, hexagons, etc. Moreover, the sorbent sheets can be shaped to be geometrically repeatable such that their outer edges are shared and that they are contiguous when duplicated.

Typical methods of preparing the sorbent sheet and spiral include compression, laser ablation, LIGA processes, photo-lithographic patterning, mechanical or chemical etching, EDM, vapor spray, laser deposition, casting, injection molding, hydroforming, stamping, extruding, silk screening, electrodeposition, electroplating, electrodeless plating, electrostatic self-assembly, atomic layer deposition, and other related or similar product techniques. Further, using these methods, the sorbent sheets and spirals can be formed either with suitable binders and/or hygroscopic materials or self-bound by the sorbent composition.

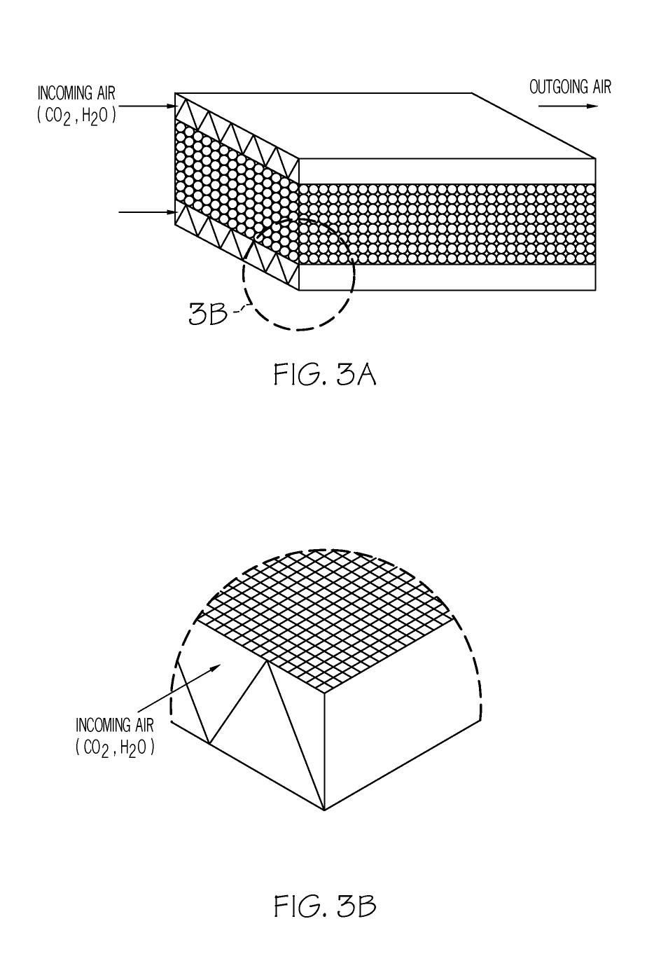

In some further embodiments of the sorbent material, as shown by FIG. 3, the substrate comprises a top layer and a bottom layer, while one or more sorbent compositions (ferrate compounds) form a sorbent bed. The top layer covers one surface of the sorbent bed while the bottom layer covers the other surface of the sorbent bed, forming a sandwich with the sorbent bed in between the top and bottom layers. The sorbent compositions can be either the raw ferrate compounds or the processed ferrate particles, such as ferrate pellets or granules. Further, this sorbent material can then be joined with a substrate to form into either a sheet or a spiral. The top and bottom layer can be a single layer or multiple layers.

According to some further embodiments, the top layer has an upper covering, one or more air spacers and a lower covering in contact with an upper surface of the sorbent bed. The air spacer separates the upper covering from the lower covering, forming air spaces or channels inside the top layer to allow passage of air streams. The bottom layer has an upper covering in contact with a lower surface of the sorbent bed, a lower covering, and one or more air spacers separating the upper covering from the lower covering, forming air spaces or channels inside the top layer to allow passages of air streams. Preferably, the air spacer has curves, ridges, corrugations, other similar shapes, or mixtures thereof.

More preferably, the upper coverings, the air spacers, and the lower coverings of the top layer and the bottom layer comprise one or more porous materials. An incoming moist air stream can flow through the upper coverings, the air spacers, the channels/air spaces formed by the air spacers, and/or the lower coverings of the top and bottom layers, and then contact the ferrate particles in the sorbent bed to generate a revitalized air stream by removing the CO₂ and co-generating O₂. The revitalized air stream may be discharged through the upper coverings, the air spacers, the channels/air spaces formed by the air spacers and/or the lower coverings of the top and bottom layers. Typical examples of the porous material include matt, screen, porous paper, woven/nonwoven fabric, perforated plastic, or a mixture thereof.

In some further embodiments, the incoming air stream enters some channels in the top and bottom layer of sorbent material, which then is absorbed by the sorbent layer. The outgoing air stream, including the revitalized air, then exits the sorbent material through other channels, which are typically different from those used by the incoming air stream. Preferably, some parts of the channels/spaces formed by air spacers can be blocked so that the incoming air stream flows through some channels while the outgoing air stream (revitalized air stream) flows out through other channels.

The applications of the above ferrate sorbent materials are also embodiments of the present invention. According to some embodiments, the present invention provides a breathing system for use in a hostile environment to absorb CO₂ and co-generate O₂, comprising:

(a) one or more breathing components to receive one or more exhaled moist air streams from one or more users, wherein the exhaled moist air stream comprises CO₂ and moisture; and

(b) one or more sorption components for absorbing CO₂ and H₂O, and to co-generate O₂, resulting in solid products and a revitalized air suitable for rebreathing, wherein the sorption component comprises one or more sorbent materials described above or elsewhere in the application.

Preferably, the breathing system is portable. The hostile environment is any environment that requires the air to be revitalized, such as underwater diving, mining, space stations, and other emergency situations. Many of these hostile environments can have temperatures ranging from room temperature to warm to hot. Such temperatures, especially hot temperatures, would prevent formation of the liquid water layer on the sorbent composition/material needed for CO₂ absorption and O₂ co-generation. One or more cooling agents and/or cooling components can be used as needed to reduce the temperatures near the sorbent composition/material. The preferred cooling agents are described in detail above in the application. The breathing system can include at least one component containing H₂O, which can provide additional water for the formation of the liquid water layer on the sorbent composition/material. The H₂O component is described above in the application. Of course, other known methods of providing H₂O can also be used. Preferably, the H₂O component is not too heavy and/or bulky especially for the portable breathing system.

In some embodiments, the breathing system includes an agitation component to shake loose the solid products from the sorbent material so as to increase the CO₂ absorption capacity of the sorbent material. Shaking should be gentle so as to avoid creating dusts or other undesirable results, such as dislodging parts of the sorbent composition unexpectedly. The agitation should be just enough to increase the reaction rate and shake the solid products off the liquid-gas interface between the surface liquid film covering the ferrate particle and the air. As such, the agitation is believed to minimize the diffusion boundary for the solid to solid conversion discussed above so that the ferrate particle would not be blocked from absorbing CO₂. Such vibration can be periodical or continuous. Agitation can also assist in reducing local heating from the exothermic process of CO₂ absorption and O₂ generation by the sorbent composition/material, and preventing the gelling or congestion of the sorbent composition/material to enable further access of H₂O and CO₂ to the sorbent composition/material for continuous CO₂ absorption and O₂ co-generation until most of or all of ferrate compounds are exhausted.

Typically, suitable vibration methods include shaking, rolling, gas sparging agitation, physical stirring, sonic/ultrasonic pulsing, and the like.

More preferably, the equipment should have compact physical designs. Such compact physical designs would place the reagents in close proximity to each other, facilitating faster reaction rates.

According to some embodiments of the breathing system, the ferrate (IV), ferrate(V) and/or ferrate(VI) compositions should be sufficiently porous or otherwise physically distributed, to provide a low pressure drop across the sorbent material or sorbent bed to enable the users to breathe easier. Examples of suitable shapes for the sorbent (ferrate) composition include coarse grains, wafers, pellets, and the like, powder layered into stack trays, and the like.

Raw ferrate compounds are usually in the shapes of amorphous powder (Fe(IV) compounds), needle shaped crystals (Fe(V) compounds), irregular granular shaped crystals (Fe(VI) compounds). Raw ferrate compounds can typically be processed using the currently known methods into forms of granules, extrudates, disks, briquettes, pellets, prills, microspheres, fiber, etc. The processed ferrate compounds can then be incorporated into a sorption component, such as a sorbent canister, sheet, and/or spiral, etc, of a breathing system.

The pressure drop across the sorbent layer of the sorption component should be low so as to enable the user to breathe easily and comfortably. Higher particle sizes of the ferrate particles can decrease the pressure drop, but they decrease the CO₂ absorption rates by decreasing available surface areas available for absorption or increasing the void spaces inside the sorbent layer. Conversely, increasing the available surface area on the sorbent layer can increase the CO₂ absorption capacity of the sorbent material. So the goal of enhancing the performance of the sorbent material is to decrease the pressure drop across the sorbent material while increasing the CO₂ absorption rate.

One way of enhancing performance is the formation of the ferrate fibers as described above. Another way is the formation of sheets, stacks, and/or spiral shapes of the ferrate sorbent material by combining the ferrate compound with one or more suitable substrate mentioned above. The substrate can be formed of any suitable porous material. Preferably, the substrate can include multiple porous layers, and may be made more porous by the inclusion of air spaces/channels formed by air spacers in the shape of curves, ridges, or corrugations.

Alternatively, a tighter bed sorbent packing can be used in combination with a pump because the pump can force the air stream from the breathing of the user to pass through the sorption bed. In a further embodiment, such pumps are synchronized with the breathing of the user to increase the efficiency of CO₂ absorption. Suitable pump typically includes an air pump, a vacuum pump, or a similar device.

According to some further embodiments, the rebreather is fitted with suitable exit gas filtration component, or equivalent, to retard any fine particulates or dust that may exit along with the outgoing revitalized air stream.

As a side note, inert dilution gas (such as N₂, Ar, He) is not affected by the ferrate sorbent or CO₂ sorption reactions. As such, the dilution gas is able to pass through the device and retain its diluent role.

Possible applications for this invention include rebreathers for scuba divers, astronauts, emergency first responders such as ambulances, police, fire fighters, miners and submarine operators during foul air events and emergency situations, and chemical plant manufacturers to work within large chemical storage tanks and reactors during cleaning and maintenance. Emergency situations typically include mine cave ins, poison gas leaks, confined space workers, poisonous gas warfare, traffic accidents with injuries, and battlefield injuries. Further, the invention can be used in space applications to supply O₂ and absorb CO₂ to maintain efficacious breathing environments for astronauts.

Another benefit is that ferrate, being a strong disinfectant, also disinfects some or all bacteria or virus from the outgoing revitalized air stream.

In some broad alternative embodiments, the present invention provides a method for absorbing CO2 and co-generating O2 using the above described sorbent composition/materials, which includes the following process:

providing one or more breathing systems containing a carbon dioxide sorbent selected from one or more sorbent compositions/materials described above,

introducing one or more streams of moist air containing CO₂ and H₂O into the breathing system; and

contacting the sorbent material with H₂O to form a liquid water layer, wherein a part or all of the ferrate compounds dissolve in the liquid water layer to absorb CO₂ and to co-generate O₂, resulting in solid products and a revitalized air suitable for rebreathing.

This method preferably has a pH in the range of about 6 to about 10, more preferably in the range of about 6.5 to about 9, and most preferably in a range of about 7 to about 8. These pH ranges reflect the process of CO₂ absorption and O₂ generation. First, a liquid water layer forms on the sorbent composition. The ferrate then dissolves in the liquid water layer, increasing pH to be very basic. The dissolution rate of the ferrate can be controlled using methods described above. CO₂ then dissolves to form carbonate or bicarbonate acids, reducing pH to neutral or slightly alkaline. The ratio of CO₂ amount to the ferrate amount can determine the rate of this pH reduction. Additional factors can also impact the rate of pH reduction, such as other basic ingredients or excipients in the sorbent composition/material, temperature etc., such as highly basic KOH. Once the pH is reduced sufficiently by CO₂, then the ferrate compound or ion will react with H₂O to generate O₂, increasing pH to 10 or higher, which can be reduced by fresh or new CO₂. Therefore, it is not desirable to have a pH buffer, or any ingredient that might act like a pH buffer, because the pH buffer might interfere with the pH changes needed for this process.

In some preferred embodiments of the method, the temperature is controlled to form a liquid water layer on the sorbent material, which is needed for the process of CO₂ absorption and O₂ co-generation. The temperature control can be accomplished through various known processes. Typically, the temperature can be controlled through the formulation(s) of the sorbent composition/material to spread the ferrate compounds, and the addition of cooling agents, both of which are described above in the application.

In addition to, or in combination with, the temperature control, the method can include a step of providing additional H₂O to enable or assist in the formation of the liquid water layer on the sorbent composition/material. The additional H₂O can be provided in many known methods, such as through hygroscopic materials, additional water vessels, etc., some of which are described above in the application.

Preferably, the method further includes a step of vibrating or agitating the sorbent material so as to increase the CO₂ absorption capacity of the sorbent material. Agitation can also assist in reducing local heating from the exothermic process of CO₂ absorption and O₂ generation by the sorbent composition/material, and preventing the gelling or congestion of the sorbent composition/material to enable further access of H₂O and CO₂ to the sorbent composition/material for continuous CO₂ absorption and O₂ co-generation until all ferrate compounds are exhausted. More preferably, the method also includes discharging the revitalized air and/or recirculating the discharged revitalized air. The process of recirculating the discharged revitalized air can produce a cleaner air more suitable for breathing by a human in case the initial process does not achieve sufficient CO₂ absorption and O₂ generation. In addition, this process can also be used to substantially disinfect the entering stream of air/atmosphere. Preferably, the ferrate particles in the sorbent material dissolve gradually and in direct proportion to the CO₂-ladened moisture introduced.

### EXAMPLES

The experimental methods used to prepare the ferrate sorbent composition and to use the sorbent composition to absorb CO₂ and co-generate O₂ are described below. These examples are provided to illustrate various embodiments of the invention and are not intended to limit the scope of the invention in any way.

### Example 1

### Method of CO₂ Absorption and O₂ Co-generation by the Neat Potassium Ferrate

This example explores the process of CO₂ absorption and O₂ co-generation by the neat potassium ferrate. The neat potassium ferrate was the 99.999% pure potassium ferrate without any other excipients or additives.

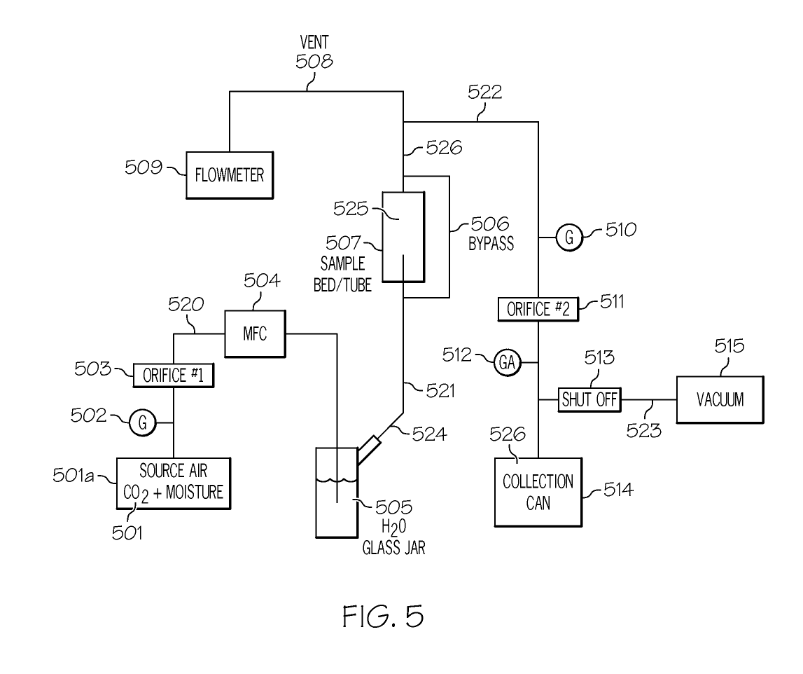

The system used for the example is illustrated in FIG. 5 without the H₂O glass jar 505. Initially, the source air 501 from the source can 501a, which includes 5% CO₂ and a high moisture level, passed through the line 520 to be bubbled through the water in the glass jar 505 to be the moisture-rich CO₂ air stream 524. For Example 1, no glass jar 505 was used, and the source air 501 did not bubble through the water or add more moisture to the air stream. Therefore, the moisture-rich CO₂ air stream 524 and the source air stream 501 were the same. The moisture-rich CO₂ air stream 524 then passed initially through the bypass 506 via the line 521, and then the majority of the air stream 524 passed through the line 522 to the collection can or to the vacuum 515 via the line 523, and a very small amount of the air stream 514 was vented through the vent 508. The vent 508 was installed to prevent any excess pressure from building up in the system. The vacuum was applied initially for a few minutes to ensure that all of the atmosphere air was evacuated from the system, and to ensure that only source air 501 and the enriched air stream 524 were in the system. Then, the shut off valve 513 was shut off, and then the vacuum 515 was turned off so that the air stream 524 flew to the collection can 514. The collection can 514 was pressurized with humid UHP nitrogen after collecting any sample. The resulting air sample was collected as the negative control sample, which was checked to see whether or not any CO₂ was lost from the system and any O₂ was leaked into the system. This negative sample was not collected for this example, but was collected for the subsequent examples. The exiting air from the vent was tested for the flow rate using the flow meter 509 to prevent any possible backflow of room air into the system.

After the collection of the negative sample, the bypass was turned off so that the air stream 524 flew through the sample tube 507 which contained the sorbent composition 525, to become the refreshed air stream 526. The refreshed air stream 526 was collected in the collection can 514. Once the collection can was filled up, the sorbent composition 525 was replaced with a fresh and/or different sorbent composition, and another sample of refreshed air stream 526 was collected. The sorbent compositions used in this example were commercially purchased Sodasorb® from WR Grace & Company, 4-8 Tyler mesh and ethyl violet indicator, lot CX10-P115-07, and the neat potassium ferrate. The sorbent compositions and the results are listed in Table 1.

During the first section of the system (from the source can 501 to the H2O glass jar 505), the system pressure was measured by the psi gauge 502 (indicated by “G”), the flow rate was controlled by orifice #1 503, and the mass flow controller 504 (“MFC”) was set to 1 L/min and was used to read the flow rate of the gas from the source 501. During the second or last section of the system (from either bypass 506 or the sample tube 507 to the vacuum 515 or to the collection can 514), the system pressure was measured by the psi gauge 510 before the orifice #2 511, the flow rate was slowed or controlled by orifice #2 511, and the post orifice system pressure was measured by the inches Hg gauge 512 (“GA”).

The source gas contained 5% CO₂ in humid ultra high purity (UHP) nitrogen. The sample tube was filled with 5 g sorbent composition/material (5 g Sodasorb® or 5 g neat potassium ferrate). When Sodasorb® was the sorbent composition in the sample tube 507, the flow rate as measured by MFC 504 started out being 0.291 L/min and finished being 0.261 L/min. The refreshed air stream 526 flew into the collection can 514 at 35 ml/min, and so it took about 10 minutes to fill out the collection can. When potassium ferrate was the sorbent composition in the sample tube 507, the flow rate started out being 0.241 L/min and finished being 0.194 L/min. The refreshed air stream 526 flew into the collection can 514 at 35 ml/min, and so it took about 10 minutes to fill out the collection can.

Before collecting any sample, the collection cans were evacuated and sealed off. After collecting any sample, all the collection cans were pressurized with humid UHP nitrogen to prevent contamination from room air. The filled collection cans 514 along with the source can 501a were tested for % CO₂ and % O₂, and the results are listed in Table 1. % O₂ was tested by HP 5890 Series II gas chromatograph with a thermal conductivity detector CTR-3 column (Grace Davison Discovery Sciences, Part #8725, SN 611020742), either 20 μl sample loop (oxygen curve 5-25%) or 250 μl sample loop (oxygen curve 0.5-2%) was used depending on the concentration of O₂ tested. In this example, 20 μl sample loop was used to test for the oxygen concentration in the range of 5-25%. % CO₂ was tested using HP 5890 Series II gas chromatograph with methanizer and flame ionization detector CTR-1 column (Grace Davison Discovery Sciences, Part #8700, SN466812W-706010497), 25 μl sample loop. All samples were injected by hand. Instrument control and data calculation were performed with GC ChemStation software (Agilent, revision A.10.02). The humidity measurement was performed with Control Company traceable hygrometer 4185, SN 101451417. The humidity was measured on the exiting air stream from the vent.

TABLE 1

| Sample ID | Description | % CO₂ | % O₂ |
|---|---|---|---|
| 1 | Source | 4.17 | 0.00 |
| 2 | Sodasorb ® | 1.52 | 0.00 |
| 3 | Potassium ferrate | 3.13 | 1.14* |

*% O₂ reading for sample 3 might be due to the leak in the system.

During the experiment, Sodasorb® in the sample tube turned lavender and warm, but cooled and turned back to white after a while. The ferrate sample got slightly warm during the experiment, and more interestingly, the ferrate sample was pushed or “blown” to the top of the sample tube when the air stream was turned on. The humidity of the exiting air from the vent ranged from 41.4% to about 70.9% depending on the flow rate of the air stream 501 from the source can 501a: The slower flow rate, the higher humidity; the faster flow rate, the lower humidity.

Some CO₂ absorption by the ferrate compounds was observed (comparing samples 1 and 3 in Table 1). However, no significant O₂ generation by the ferrate compound was found. The 1.14% O₂ reading for sample 3 might be due to a leak in the system or it can be generated by the ferrate compound. However, since the 1.14% O₂ level was below the lower detection limit of 5%, the O₂ level was not very reliable. A more sensitive O₂ testing needed for detecting a lower level of O₂. Moreover, a negative control sample would be collected in the subsequent examples to ensure that the system had no leak.

More importantly, the refreshed air stream sample was collected within a few minutes of exposing the neat ferrate particles to CO₂ and H₂O, the ferrate might not have time to react to generate O₂ yet because the pH might not be reduced sufficiently to be suitable for generation of O₂. It is also possible that the temperature in the lab was too high for condensation of moisture to form the liquid water layer on the ferrate compounds.

### Example 2

### Method of CO₂ Absorption and O₂ Co-Generation by the Neat Potassium Ferrate

This example explores the process of CO₂ absorption and O₂ co-generation by the neat potassium ferrate. The neat potassium ferrate was the 99.999% pure potassium ferrate without any other excipients or additives.

The system used for the example is illustrated in FIG. 5 without the H₂O glass jar 505, and is described in Example 1. Moreover, a negative air stream sample was collected to ensure the system had no leakage. The oxygen was measured using 20 μl sample loop, which used oxygen curve of 5-25%. All other processes, parameters, system set ups and equipment were the same as that of Example 1. The sorbent compositions and the CO₂ and O₂ results are listed in Table 2.

The same Sodasorb® and potassium ferrate sample tubes were tested twice: once in the morning and another in the afternoon. The test results from the morning experimental collection cans are listed as Set 1 data in Table 2, while the test results from the afternoon are listed as Set 2 data in Table 2. The humidity of the air stream at the exit valve was the same as that of Example 1. The flow rates were controlled by the pressure, and the actual flow rate readings varied from 0.147 L/min to about 0.316 L/min.

TABLE 2

| | | | |
|---|---|---|---|
| Sample ID | Description | % CO₂ | % O₂ |

Set 1

| | | | |
|---|---|---|---|
| 4 | Source | 6.29 | 0 |
| 5 | Negative control | 6.45 | 0 |
| 6 | Sodasorb ® | 2.12 | 0 |
| 7 | Potassium ferrate | 2.23 | 0 |

Set 2

| | | | |
|---|---|---|---|
| 8 | Source | 4.72 | 0 |
| 9 | Negative control | 5.50 | 0 |
| 10 | Sodasorb ® | 2.73 | 0 |
| 11 | Potassium ferrate | 5.15 | 0 |

Comparing the data from Set 1, both Sodasorb® and potassium ferrate showed significant and comparable CO₂ absorption, but both sorbent compositions showed no O₂ generation. The negative control readings for both CO₂ and O₂ level are similar to that of the source, suggesting no leakage for the system during the experiment. No color change was observed for the ferrate sample tube. While not wishing to be bound by theory, it is presently believed that insufficient water was available for the ferrate to react to generate O₂. It is also possible that the temperature in the lab was too high for condensation of moisture to form the liquid water layer on the ferrate compounds. The addition of a hygroscopic material might resolve the problem.

### Example 3

### Method of CO₂ Absorption and O₂ Co-Generation by the Mixture of Ferrate and Hygroscopic Material

This example explores the process of CO₂ absorption and O₂ co-generation by a mixture of the ferrate compound and hygroscopic material, comparing to that of neat ferrate compound. The hygroscopic materials were dry KOH powder and K₃PO₄ particles. KOH was grounded into powder under argon prior to being mixed with the ferrate compound.

The neat ferrate compound was 99.999% pure potassium ferrate without any other excipients or additives. Two mixtures of ferrate/hygroscopic samples were made: One was the ferrate/KOH mixture (“ferrate/KOH”), in which 2.5 g of pure potassium ferrate (same as the neat ferrate compound) and 2.5 KOH grounded powder were mixed together by a spatula for a few minutes. The other was the ferrate/K₃PO₄ mixture (“ferrate/K₃PO₄”), in which 2.5 g potassium ferrate was mixed with 2.5 g K₃PO₄ by a spatula for a few minutes.

The system used for the example is illustrated in FIG. 5 with the H2O glass jar 505, and is described in Example 1. Moreover, a negative air stream sample was collected to ensure the system had no leakage. The oxygen was measured using 250 μl sample loop, which used oxygen curve of 0.5-2%, resulting in a detection sensitivity of 0.5 to 2%. The MFC 504 were set to 0.125 L/min, and the additional H₂O was introduced into the incoming air stream by bubbling the source air 501 through the H₂O in the H₂O glass jar 505, resulting in the moisture-rich CO₂ air stream 524. All other processes, parameters, system set ups and equipment were the same as that of Example 1. The humidity level of the exiting air stream from the vent was 83.5% and above. The sorbent compositions and the CO₂ and O₂ results are listed in Table 3.

TABLE 3

| Sample ID | Description | % CO₂ | % O₂ |
|---|---|---|---|
| 12 | Negative control #1 | 3.92 | 1.64 |
| 13 | Ferrate | 1.34 | 1.51 |
| 14 | Ferrate/KOH | 0.30 | 0.42 |
| 15 | Ferrate/K₃PO₄ | 3.51 | 0.60 |
| 16 | Negative control #2 | 5.36 | 0.48 |
| 17 | KOH | 1.02 | 0.51 |
| 18 | K₃PO₄ | 5.00 | 0.48 |
| 19 | Source #1 | 5.54 | 0.00 |
| 20 | Source #2 | 6.24 | 0.00 |

After collecting the refreshed air stream samples in the collection can for each sample compositions, the sample tubes were stored in desiccators. One or two days later, the samples from the sample tubes were examined. It was found that most of the left over ferrate compounds are active ferrates. No color changes were observed for the ferrate sample tubes. It was believed that too much KOH and too much K₃PO₄ were added so that the pH was too high for the ferrate compound to generate O₂ within the short period of time (10-12 minutes of collecting the exiting air sample). It would take a very long time for CO₂ to overcome the alkalinity so as to enable the ferrate to generate O₂. So the amount of hygroscopic material should be reduced to about 1-5 wt %.

Moreover, the examination of the used samples suggested that additional moisture and CO₂ should be introduced into the system. Therefore, the CO₂ level in the source can 501a should be increased, and the flow rate of the incoming air stream be slowed so as to allow time for the moisture and CO₂ to be absorbed by the sorbent composition in the sample tube.

### Example 4

### Method of CO₂ Absorption and O₂ Co-Generation by the Mixture of Ferrate and a Lower Level of KOH

This example explores the process of CO₂ absorption and O₂ co-generation by a mixture of the ferrate compound and 3 wt % KOH, using 20% CO₂ in the source air stream and the low flow rate of 0.050 L/min. KOH used was 50% KOH solution.

The neat ferrate compound was 99.999% pure potassium ferrate without any other excipients or additives.

The mixture of ferrate/KOH was made by the following method:
1. 6.563 g potassium ferrate and 0.437 g 50% KOH (KOH in water) were weighed out. Comparing the wt % of ferrate to that of KOH, the ferrate was 97 wt % and KOH was about 3 wt %. The mixture contained water. When water was taken into account, the mixture contained about 3.44 wt % water, 2.81 wt % KOH, and 93.75 wt % ferrate.
2. The ferrate and KOH solution were mixed and milled for an hour in a mill jar containing small glass beads at 140 rpm with periodic gentle shaking to dislodge the mixture from sticking to the jar.
3. The resulting mixture had a consistency of brown sugar.

The system used for the example is illustrated in FIG. 5 with the H₂O glass jar 505, and is described in Example 1. The source air was 20% CO₂ in humid UHP nitrogen. Moreover, a negative air stream sample was collected to ensure the system had no leakage. The oxygen was measured using 250 μl sample loop, which used oxygen curve of 0.5-2%, resulting in a detection sensitivity of 0.5 to 2%.

The MFC 504 were set to 0.050 L/min, and the additional H₂O was introduced into the incoming air stream by bubbling the source air 501 through the H₂O in the H₂O glass jar 505, resulting in the moisture-rich CO₂ air stream 524. All other processes, parameters, system set ups and equipment were the same as that of Example 1. The humidity level of the exiting air stream from the vent was 83.5% and above.

Five cans of refreshed air streams (“ferrate/KOH 1 to 5”) were collected from the same ferrate composition—the mixture of ferrate and KOH (“ferrate/KOH”) at the different time interval. However, the 5^{th} can (“ferrate 5”) was accidentally pressurized with room air instead of the desired nitrogen, and therefore, the results from the ferrate 5 are not correct.

The sorbent compositions and the CO₂ and O₂ results are listed in Table 4. In Table 4, “collection time” is based on the total time need to collect the exiting refreshed air sample. It took generally 15 minutes to collect a can of exiting air sample, so ferrate/KOH 1 had 15 minutes of collection time and ferrate/KOH 2 had 30 minutes of collection time because it was collected immediately after the collection of ferrate/KOH 1. The collection time for ferrate 3 had 55 minutes collection time because there was a 10 minute wait time after the collection of ferrate/KOH 2: collection time for ferrate/KOH 3=10 minutes wait+15 minutes of collection for ferrate/KOH 3+30 minutes of collection time for ferrate/KOH 2.

The top of the ferrate/KOH sample tube turned warmer about 20-30 minutes into the experiment (during the collection of the ferrate/KOH 2). After collecting all the air samples, the ferrate/KOH mixture needed to be pushed out the sample tube with some force, while in the previous examples, the ferrate composition easily came out of the sample tube after light tapping. Before the experiment, the ferrate/KOH mixture had a consistency and color of dark brown sugar. After the experiment, the ferrate/KOH mixture (“the used ferrate/KOH mixture”) was much drier, with some browning along the side of the sample tube while most of the ferrate/KOH mixture did not change color. In addition, lots of ferrate activities left in the used ferrate/KOH mixture.

TABLE 4

| Sample | | Collection Time | | |
|---|---|---|---|---|
| ID | Description | (min) | % CO₂ | % O₂ |
| 21 | Negative control | n/a | 18.49 | 0.81 |
| 22 | Ferrate/KOH 1 | 15 | 4.10 | 4.30 |
| 23 | Ferrate/KOH 2 | 30 | 2.80 | 4.02 |
| 24 | Ferrate/KOH 3 | 55 | 17.44 | 0.57 |
| 25 | Ferrate/KOH 4 | 90 | 24.42 | 0.69 |
| 26 | Ferrate/KOH 5 | 120* | 23.42 | 5.78 |
| 27 | Source | n/a | 24.25 | 0.00 |

*Ferrate/KOH 5 data is not reliable because the collection can was accidentally pressurized with room air instead of nitrogen.

The results from Table 4 show that the ferrate/KOH mixture showed significant CO₂ absorption and O₂ co-generation for the first 30 minutes. T reactions associated with the CO₂ absorption and O₂ co-generation appeared to be exothermic, which might result in local heating of the ferrate/KOH mixture in the sample tube. After about 30 minutes of reaction time, the local heating within the congested space of the sample tube might be sufficient to generate a barrier against any future CO₂ and/or H₂O absorption by the sorbent composition. While not wishing to be bound by theory, it is currently believed that the heating prevented the condensation of water vapor from the incoming air stream on the ferrate sorbent composition, drying out the ferrate/KOH mixture. It is suggested that the combination of spreading out of the ferrate sorbent composition and the cooling agent/equipment might resolve this issue.

### Example 5

### Method of CO₂ Absorption and O₂ Co-Generation by the Mixture of Ferrate and KOH Using 100% CO₂ as the Source Air Stream

This example explores the process of CO₂ absorption and O₂ co-generation by a mixture of the ferrate compound and 3 wt % KOH, using 100% CO₂ in the source air stream and the low flow rate of 0.030-0.050 L/min. KOH used was 50% KOH solution.

The neat ferrate compound was 99.999% pure potassium ferrate without any other excipients or additives. The mixture of ferrate/KOH was the same as that of Example 4. All other processes, parameters, system set ups and equipment were the same as that of Example 4. The humidity level of the exiting air stream from the vent was 83.5% and above.

Two cans of refreshed air streams (“ferrate/KOH 1 and 2”) were able to be collected from the same ferrate composition—the mixture of ferrate and KOH (“ferrate/KOH”) at the different time interval. The sorbent compositions and the CO₂ and O₂ results are listed in Table 5.

TABLE 5

| Sample | | Collection Time | | |
|---|---|---|---|---|
| ID | Description | (min) | % CO₂ | % O₂ |
| 28 | Negative control | n/a | 119.90 | 0.50 |
| 29 | Ferrate/KOH 1 | 15 | 119.28 | 5.27 |
| 30 | Ferrate/KOH 2 | 30 | 127.77 | 0.41 |
| 31 | Source | n/a | 142.73 | 0.00 |

The ferrate/KOH sample tube turned really hot almost immediately after starting the experiment (without about 10-15 minutes). The ferrate/KOH mixture was separated into three sections. It was believed that O₂ generated from the mixture might have created these sections. The data in Table 5 shows that there was definite O₂ generation by the ferrate/KOH mixture for the first 15 minutes. However, the results do not show any CO₂ absorption. It is possible that CO₂ absorption occurred during the first couple of minutes, which might not show up in the collective air sample after 15 minutes, especially when the variation for the CO2 level was in the range of about 23% (the variation is based on the difference in CO₂ between that of the negative control and that of the source).

While not wishing to be bound by theory, it is currently believed that the heating prevented the condensation of water vapor from the incoming air stream on the ferrate sorbent composition, drying out the ferrate/KOH mixture. It is suggested that the combination of spreading out of the ferrate sorbent composition and the cooling agent/equipment might resolve this issue.

While the forms of the invention herein disclosed constitute presently preferred embodiments, many others are possible. It is not intended herein to mention all of the possible equivalent forms or ramifications of the invention. It is to be understood that the terms used herein are merely descriptive, rather than limiting, and that various changes may be made without departing from the spirit of the scope of the invention.

1. A sorbent composition suitable for removal of CO₂ and co-generation of O₂, comprising Fe(IV), Fe(VI), Fe(V), or a mixture thereof (“the ferrate compound”), wherein upon exposure to CO₂ and H₂O, the sorbent composition is capable of absorbing CO₂ and co-generating O₂.

2. The sorbent composition of claim 1, wherein the sorbent composition is in the form of granule, extrudate, sphere, disk, briquette, pellet, prill, solid solution, microsphere, encapsulate, or a mixture thereof.

3. The sorbent composition of claim 1, further comprising one or more hygroscopic materials.

4. The sorbent composition of claim 1, further comprising H₂O.

5. The sorbent composition of claim 1, further comprising one or more cooling agents.

6. A sorbent material suitable for removal of CO₂ and co-generation of O₂, comprising:
one or more sorbent compositions of claims 1 to 5.

7. The sorbent material of claim 6, wherein the sorbent compositions are embedded in one or more fibers.

8. The sorbent material of claim 6,
wherein one or more sorbent compositions join with one or more substrates to form a sorbent layer.

9. The sorbent material of claim 8, wherein the sorbent compositions are coated on one or more substrates.

10. The sorbent material of claim 8, wherein the substrate comprises one or more mats, beads, screens, porous material (paper, fabric or plastic), perforated plastic, perforated and corrugated plastic, woven fabric, non-woven fabric, or mixtures thereof.

11. The sorbent material of claim 8, wherein the substrate comprises one or more hygroscopic materials.

12. The sorbent material of claim 8,
wherein the substrate comprises a top layer and a bottom layer,
wherein one or more sorbent compositions form a sorbent layer; and
wherein the top layer covers one surface of the sorbent bed and the bottom layer covers the other surface of the sorbent bed.

13. The sorbent material of claim 12,
wherein the top layer has an upper covering, one or more air spacers and a lower covering in contact with an upper surface of the sorbent bed, wherein the air spacers separate the upper covering from the lower covering, forming channels inside the top layer; and
wherein the bottom layer has an upper covering in contact with a lower surface of the sorbent bed, a lower covering, and one or more air spacers separating the upper covering from the lower covering, forming channels inside the top layer.

14. The sorbent material of claim 13, wherein the upper coverings, the air spacers, and the lower coverings of the top layer and the bottom layer comprise one or more porous materials.

15. The sorbent material of claim 14, wherein the porous material comprises matt, screen, porous paper, woven/nonwoven fabric, perforated plastic, or a mixture thereof.

16. The sorbent materials of claims 6 to 15, wherein the sorbent compositions disinfect the incoming air and/or the revitalized air.

17. A breathing system for use in a hostile environment to absorb CO₂ and co-generate O₂, comprising
(a) one or more breathing components to receive one or more exhaled moist air streams from one or more users, wherein the exhaled moist air stream comprises CO₂ and moisture; and
(b) one or more sorption components for absorbing CO₂ and H₂O, and to co-generate O₂, resulting in solid products and a revitalized air suitable for rebreathing, wherein the sorption component comprises one or more sorbent materials of claims 6 to 16.

18. The breathing system of claim 17, further comprising one or more cooling agents and/or cooling components.

19. The breathing system of claim 17, further comprising at least one component comprising H₂O.

20. The breathing system of claim 17, further comprising at least one agitation component to shake loose the solid products from the sorbent material.

21. The breathing system of claim 17, further comprising at least one pump to drive the incoming air stream through the sorbent layer, wherein the pump comprises an air pump, a vacuum pump, or a similar device.

22. The breathing system of claim 17, further comprising at least one suitable exit gas filtration component so as to prevent any fines or dust from exiting with the revitalized air.

23. The breathing system of claim 17, wherein the system can be used as a rebreather underwater, as emergency first responders, in mining, and in other emergency situations.

24. The breathing system of claim 17, wherein the system is portable.

25. A method for absorbing CO₂ and co-generating O₂, comprising the steps of:
(a) providing one or more breathing systems of claims 17 to 24;
(b) introducing one or more streams of moist air containing CO₂ and H₂O into the breathing system; and
(c) contacting the sorbent material with H₂O to form a liquid water layer, wherein a part or all of the ferrate compounds dissolve in the liquid water layer to absorb CO₂ and to co-generate O₂, resulting in solid products and a revitalized air suitable for rebreathing.

26. The method of claim 25, wherein pH is in a range of about 6 to about 10, preferably in a range of about 6.5 to about 9, and most preferably in a range of about 7 to about 8.

27. The method of claim 25, wherein the temperature is controlled to enable or assist in the formation of a liquid water layer on the sorbent material.

28. The method of claim 25, further comprising a step of providing an additional H₂O.

29. The method of claim 25, further comprising a step of shaking loose some or all of the solid products from the sorbent material.

30. The method of claim 25, further comprising steps of discharging the revitalized air and/or recirculating the discharged revitalized air.
